(19) United States
(12) Patent Application Publication
(10) Pub. No.: US 2025/0093319 A1
(43) Pub. Date: Mar. 20, 2025

(54) DETECTING WINE CHARACTERISTICS FROM WINE SAMPLES

(71) Applicant: Ye Feng, Portland, OR (US)

(72) Inventor: Ye Feng, Portland, OR (US)

(21) Appl. No.: 18/961,351

(22) Filed: Nov. 26, 2024

Related U.S. Application Data
(63) Continuation-in-part of application No. PCT/US2023/020952, filed on May 4, 2023.
(60) Provisional application No. 63/346,787, filed on May 27, 2022.

(51) Int. Cl. G01N 33/14; G01N 21/65; G06N 3/0455; G16C 20/20; G16C 20/70

(52) U.S. Cl. CPC G01N 33/146; G01N 21/658; G06N 3/0455; G16C 20/20; G16C 20/70

(57) ABSTRACT

Methods of predicting perceptual characteristics may include: (a) receiving an optical signal from a sample of a grape product or a wine; (b) extracting features from the optical signal by transforming information in the optical signal to a latent space; and (c) providing the features to a machine learning model that outputs (i) chemical composition information in the grape product or the wine, and/or (ii) the one or more perceptual characteristics of a finished wine produced from the grape product or of the wine. The chemical composition information and/or the perceptual characteristics may pertain to smoke taint.

### INCORPORATION BY REFERENCE

This application is a Continuation-in-part of PCT application No. PCT/US2023/020952, entitled “DETECTING WINE CHARACTERISTICS FROM WINE SAMPLES” filed May 4, 2023, which claims priority to U.S. Provisional application No. 63/346,787, entitled “DETECTING SMOKE TAINT IN WINE SAMPLES”, filed May 27, 2022. Each application that the present application claims benefit of or priority to is incorporated by reference herein in its entirety and for all purposes.

### BACKGROUND

Wines and related spirits have complex flavor profiles that depend on the presence and relative amounts of various chemical compounds. Small changes in composition may have a big impact on flavor or other perceptual characteristics. The presence of certain uncommon compounds has been observed to produce highly undesirable perceptual characteristics such as smoke taint.

Background and contextual descriptions contained herein are provided solely for the purpose of generally presenting the context of the disclosure. Much of this disclosure presents work of the inventors, and simply because such work is described in the background section or presented as context elsewhere herein does not mean that such work is admitted prior art.

### SUMMARY

Aspects of this disclosure pertain to methods of predicting smoke taint. Such methods may be characterized by the following operations: (a) receiving an optical signal from a sample of a grape product or a wine; (b) extracting features from the optical signal by transforming information in the optical signal to a latent space; and (c) providing the features to a machine learning model that provides (i) chemical composition information in the grape product or the wine, where the chemical composition information includes information about one or more compounds that are associated smoke taint, and/or (ii) one or more perceptual characteristics of a finished wine produced from the grape product or of the wine. In these aspects, the perceptual characteristics indicate whether the finished wine produced from the grape product or the wine exhibits a smoke taint perceptual characteristic. In these aspects, the latent space has reduced dimensions compared to dimensions of the optical signal. The optical signal may comprise a spectrum having characteristics influenced by chemical components of the grape product or the wine. The method may additionally include obtaining the optical signal from the sample comprising the grape product or the wine.

In certain embodiments, the optical signal comprises information from at least 10 wavelengths. In certain embodiments, the optical signal is a signal from Raman spectroscopy performed on the grape product or the wine. The Raman spectroscopy may be a Surface Enhanced Raman Spectroscopy (SERS). In such embodiments, the method may include contacting the sample with a nanoscale material that enhances Raman signals from the sample. In some cases, the method additionally includes contacting the sample with multiple nanostructures and obtaining a Raman signal for each nanostructure and sample combination.

In certain embodiments, the sample comprises one or more grapes used to produce the finished wine. In certain embodiments, the sample comprises must or juice used to produce the finished wine. In certain embodiments, the sample comprises the wine.

In certain embodiments, the methods include providing information from a portion of a grape plant that is not a grape to the machine learning model, wherein the grape plant produced grapes for the grape product or the wine. As an example, the portion of the grape plant may be a leaf of the grape plant.

In certain embodiments, the methods include providing information from infrared, fluorescence, and/or visible spectra of the grape product or the wine to the machine learning model. In certain embodiments, the methods include providing information from an electronic sensor of volatile organic compounds to the machine learning model.

In certain embodiments, the methods include providing information about a wine making process for producing the wine or the finished wine to the machine learning model. As examples, the information about the wine making process may include grape sources, harvest time, crushing conditions, must handling, time between crushing and fermentation, temperature prior to, during or after fermentation or incubation, incubation period, yeast, inoculum size, additives, pH, substrate concentration, and any combinations thereof.

In certain embodiments, extracting features comprises providing the optical signal to a variational autoencoder or a transformer model trained using training data comprising training optical signals from smoke tainted grape products or wines.

In certain embodiments, the machine learning model is trained using training data comprising training mass spectrometry data obtained from smoke tainted grape products or wines. In such embodiments, the training mass spectrometry data may be obtained using a technique selected from a group consisting of gas chromatography-mass spectroscopy (GC-MS), gas chromatography-tandem mass spectroscopy (GC-MS-MS), high performance liquid chromatography-mass spectroscopy (HPLC-MS), high performance liquid chromatography-tandem mass spectroscopy (HPLC-MS-MS), high performance liquid chromatography-diode array detector-mass spectroscopy (HPLC-DAD-MS), and any combinations thereof. In certain embodiments, the machine learning model provides chemical composition information in the grape product or the wine corresponding to the training mass spectrometry data.

In certain embodiments, the methods include the following operations: producing multiple optical signals from the sample; extracting features from each of the multiple optical signals; and combining the features from each of the multiple optical signal to produce one or more combined features of the multiple optical samples. In some cases, providing the features to the machine learning model comprises providing the combined features of the multiple optical samples to the machine learning model.

In certain embodiments, the machine learning model is a neural network. In certain embodiments, the methods include preprocessing the optical signal to normalize and/or reduce noise in the optical signal prior to extracting features from the optical signal.

In certain embodiments, the chemical composition information includes information about a catechin, a tannin, an anthocyanin, a quercetin, a guaiacol, a cresol, a syringol, a glycoside of any of the foregoing, and any combination of the foregoing. In certain embodiments, the one or more compounds that are associated with smoke taint comprise a guaiacol, a cresol, a syringol, a glycoside of any of the foregoing, and any combination of the foregoing.

Any combination of the features mentioned for these aspects may be implemented together in methods of predicting smoke taint in accordance with this disclosure.

Some aspects of this disclosure pertain to methods of training a machine learning model configured to predict smoke taint in a wine. Such methods may be characterized by the following operations: (1) receiving training data for each of a plurality of samples, each sample comprising a grape product and/or a wine, wherein the training data for each sample comprises (a) an optical signal generated from the sample, and (b) (i) chemical composition information in the grape product, a finished wine produced from the grape product, or the wine, wherein the chemical composition information includes information about one or more compounds that are associated with smoke taint, and/or (ii) one or more perceptual characteristics of the finished wine produced from the grape product or of the wine; (2) training a feature extractor using at least a portion of the training data, wherein the feature extractor is trained to extract features from the optical signals generated from the training samples by transforming information in the optical signals to a latent space; and (3) training a machine learning model, using at least a portion of the training data and at least features extracted from the training data by the feature extractor, wherein the machine learning model is trained to predict (i) the chemical composition information in the grape product, the finished wine produced from the grape product, or the wine, and/or (ii) one or more perceptual characteristics of the finished wine produced from the grape product or of the wine.

In these aspects, the perceptual characteristics indicate whether the finished wine produced from the grape product, or the wine exhibits a smoke taint perceptual characteristic. In these aspects, the latent space has reduced dimensions compared to dimensions of the optical signals.

In certain embodiments, the plurality of samples comprises a plurality of grapes, must, and/or juice. In certain embodiments, the plurality of samples comprises a grape product spiked with one or more smoke taint compounds and/or a wine spiked with one or more smoke taint compounds.

In certain embodiments, the optical signals from the samples each comprise information from at least 10 wavelengths. In certain embodiments, the optical signals from the samples comprise Raman spectra. The Raman spectroscopy may be a Surface Enhanced Raman Spectroscopy (SERS). In such embodiments, the method may include contacting the sample with a nanoscale material that enhances Raman signals from the sample. In some cases, the method additionally includes contacting the sample with multiple nanostructures and obtaining a Raman signal for each nanostructure and sample combination.

In certain embodiments, the training data for each of the plurality of samples includes information from a portion of a grape plant that is not a grape, wherein the grape plant produced grapes for the grape product or the wine. In some cases, the portion of the grape plant comprises a leaf of the grape plant.

In certain embodiments, the training data includes information from fluorescence spectra, infrared spectra and/or visible spectra of the grape produce or the wine. In certain embodiments, the training data includes information from an electronic sensor of volatile organic compounds.

In certain embodiments, training the feature extractor comprises training a variational autoencoder or a transformer model using the optical signals generated from the samples. In certain embodiments, the machine learning model is a neural network.

In certain embodiments, the chemical composition information comprises information about a catechin, a tannin, an anthocyanin, a quercetin, a guaiacol, a cresol, a syringol, a glycoside of any of the foregoing, and any combination of the foregoing. In certain embodiments, the one or more compounds that cause smoke taint comprise a guaiacol, a-cresol, a syringol, a glycoside of any of the foregoing, and any combination of the foregoing.

Any combination of the features mentioned for these aspects may be implemented together in methods of training a machine learning model in accordance with this disclosure.

Further aspects of this disclosure pertain to methods of predicting perceptual characteristics, which are not necessarily indicative of or relate to smoke taint. Such methods may be characterized by the following operations: (a) receiving an optical signal from a sample of a grape product or a wine; (b) extracting features from the optical signal by transforming information in the optical signal to a latent space; and (c) providing the features to a machine learning model that outputs (i) chemical composition information in the grape product or the wine, and/or (ii) the one or more perceptual characteristics of a finished wine produced from the grape product or of the wine.

In these methods, the optical signal comprises a spectrum having characteristics influenced by chemical components of the grape product or the wine. In these methods, the latent space has reduced dimensions compared to dimensions of the optical signal. In these methods, the chemical composition information includes information about one or more compounds that are associated with one or more perceptual characteristics.

In certain embodiments, the one or more perceptual characteristics comprise a perceptual characteristic selected from the group consisting of a taste, an aroma, a mouthfeel, an appearance, and any combinations thereof. In some cases, the one or more perceptual characteristics comprise a smoke taint perceptual characteristic.

In certain embodiments, the one or more perceptual characteristics comprise a perceptual characteristic associated with one or more chemical byproducts of an organism; the chemical composition information comprises information about the one or more chemical byproducts of the organism. In some cases, the organism comprises Brettanomyces, and the chemical composition information comprises information about 4-ethylphenol, 4-ethylguaiacol, 4-ethylcatechol and/or 4-propylguaiacol. The methods may comprise repeating the method for multiple samples obtained at multiple stages in a wine making process. For examples, some methods may comprise using the output of the machine learning model at the multiple stages of the wine making process to account for potential variations in the presence or concentration of Brettanomyces during the wine making process.

In certain embodiments, the one or more perceptual characteristics comprise a perceptual characteristic indicating whether or not consumers favorably perceives the finished wine produced from the grape product or the wine. In certain embodiments, the one or more perceptual characteristics comprise a favorability score indicating how favorably consumers consider the finished wine produced from the grape product or the wine. In certain embodiments, the one or more perceptual characteristics comprise one or more metrics of the finished wine or a score representing the one or more metrics of the finished wine.

In certain embodiments, the machine learning model is configured to output information about grape variety, terroir details, appellation, vineyard, harvest year, additives, and other characteristics or properties of a finished wine or a grape product, and any combinations thereof.

In certain embodiments, the machine learning model is configured to output a recommendation of one or more wine making process parameters. In certain embodiments, the one or more wine making process parameters relates to grape sources, harvest time, crushing conditions, must handling, time between crushing and fermentation, temperature prior to, during or after fermentation or incubation, incubation period, yeast, inoculum size, additives, pH, substrate concentration, and any combinations thereof. In certain embodiments, the machine learning model is configured to output a recommendation of a type and/or a parameter of flavor engineering process.

In certain embodiments, the chemical composition information comprises information about a catechin, tannin, anthocyanin, terpinol, linalool, geraniol, α-terpineol, citronelol, nerol, nor-isoprenoid, β-damsascenone, β-ionone, α-ionone, ethyl cinnamate, ethyl dihydrocinnamate, hexanol, Z-3-hexenol, E-2-hexenol, ethanol, fusel alcohol, isobutanol, 2 and 3-methylbutanol, isoamylalcohol, β-phenylethanol, methionol, fusel alcohol acetate, isobutyl acetate, isoamyl acetate, hexyl acetate, phenylethyl acetate, fatty acid, acetic acid, butyric acid, hexanoic acid, octanoic acid, decanoic acid, ethyl acetate, ethyl butyrate, ethyl hexanoate, ethyl octanoate. ethyl decanoate, isobutyric acid, 2-methylbutyric acid, 3-methylbutyric acid, isovaleric acid, ethyl isobutyrate, ethyl 2-methylbutyrate, ethyl 3-methylbutyrate, ethyl isovalerate, carbonyl, lactone, diacetyl, 2,3-pentanedione, acetoine, γ-butyrolactone, ethyl lactate, diethyl succinate, Z-whiskylactone, E-whiskylactone, o and m-cresol, guaiacol, 4-methylguaiacol, eugenol, E-isoeugenol, 2,6-dimethoxyphenol, 4-allyl-2,6-dimethoxyphenol, vanillin, acetovanillone, propiovanillone, ethylvanillate, methylvanillate, furfural, 5-methylfurfural, 4-ethylphenol, 4-ethylguaiacol, 4-propylguaiacol, γ-lactones, γ-octalactone, γ-nonalactone, γ-decalactone, γ-undecalactone, γ-dodecalactone, 4-vinylphenol, 4-vinylguaiacol, and any combination of the foregoing.

In certain embodiments, the sample comprises one or more grapes, must, and/or juice used to produce the finished wine. In certain embodiments, the sample comprises the wine.

Some methods additionally include obtaining the optical signal from the sample comprising the grape product or the wine.

In certain embodiments, the optical signal is a signal from Raman spectroscopy performed on the grape product or the wine. In some cases, the Raman spectroscopy is a Surface Enhanced Raman Spectroscopy (SERS). In some examples, methods include contacting the sample with a nanoscale material that enhances Raman signals from the sample.

In certain embodiments, methods include providing information from an electronic sensor of volatile organic compounds to the machine learning model. In certain embodiments, methods include providing information from infrared, fluorescence, and/or visible spectra of the grape produce or the wine to the machine learning model.

In certain embodiments, extracting features comprises providing the optical signal to a variational autoencoder or a transformer model trained using training optical signals from training data. In certain embodiments, the machine learning model is a neural network. In certain embodiments, extracting features comprises providing the optical signal to a variational autoencoder or a transformer model trained using training data comprising training optical signals from training samples of grape products and/or wines.

In certain embodiments, the machine learning model is trained using training data comprising training mass spectrometry data obtained from training samples of grape products and/or wines. As examples, the training mass spectrometry data may be obtained using a technique selected from a group consisting of gas chromatography-mass spectroscopy (GC-MS), gas chromatography-tandem mass spectroscopy (GC-MS-MS), high performance liquid chromatography-mass spectroscopy (HPLC-MS), high performance liquid chromatography-tandem mass spectroscopy (HPLC-MS-MS), high performance liquid chromatography-diode array detector-mass spectroscopy (HPLC-DAD-MS), and any combinations thereof. In certain embodiments, the machine learning model provides chemical composition information in the grape product or the wine corresponding to the training mass spectrometry data.

In certain embodiments, the machine learning model is trained using training data comprising microbiological information indicating the presence of a microorganism. In some cases, the microbiological information is PCR results indicating the presence of the microorganism.

Any combination of the features mentioned for these aspects may be implemented together in methods of predicting perceptual characteristics in accordance with this disclosure.

Aspects of this disclosure pertain to methods of training a machine learning model configured to predict perceptual characteristics of a wine. The methods may be characterized by the following operations: (1) receiving training data for each of a plurality of samples, each sample comprising a grape product and/or a wine, wherein the training data for each sample comprises (a) an optical signal generated from the sample, and (b) (i) chemical composition information in the grape product, a finished wine produced from the grape product, or the wine, wherein the chemical composition information includes information about one or more compounds that are associated with one or more perceptual characteristics, and/or (ii) the one or more perceptual characteristics of the finished wine produced from the grape product or of the wine; (b) training a feature extractor using at least a portion of the training data, wherein the feature extractor is trained to extract features from the optical signals generated from the training samples by transforming information in the optical signals to a latent space, which latent space has reduced dimensions compared to dimensions of the optical signals; and (c) training a machine learning model, using at least a portion of the training data and at least features extracted from the training data by the feature extractor, wherein the machine learning model is trained to predict (i) the chemical composition information in the grape product, the finished wine produced from the grape product, or the wine, and/or (ii) the one or more perceptual characteristics of the finished wine produced from the grape product or of the wine.

In certain embodiments, the one or more perceptual characteristics comprise a perceptual characteristic selected from the group consisting of a taste, an aroma, a mouthfeel, an appearance, and any combinations thereof. In certain embodiments, the one or more perceptual characteristics comprise a smoke taint perceptual characteristic. In certain embodiments, the one or more perceptual characteristics comprise a Brettanomyces taint perceptual characteristic.

In certain embodiments, the one or more perceptual characteristics comprise a perceptual characteristic indicating whether the finished wine produced from the grape product or the wine is favorably perceived by consumers. In certain embodiments, the one or more perceptual characteristics comprise a favorability score indicating how favorably the finished wine produced from the grape product or the wine is perceived by consumers.

In certain embodiments, the chemical composition information comprises information about a catechin, tannin, anthocyanin, terpinol, linalool, geraniol, α-terpineol, citronelol, nerol, nor-isoprenoid, β-damsascenone, β-ionone, α-ionone, ethyl cinnamate, ethyl dihydrocinnamate, hexanol, Z-3-hexenol, E-2-hexenol, ethanol, fusel alcohol, isobutanol, 2 and 3-methylbutanol, isoamylalcohol, β-phenylethanol, methionol, fusel alcohol acetate, isobutyl acetate, isoamyl acetate, hexyl acetate, phenylethyl acetate, fatty acid, acetic acid, butyric acid, hexanoic acid, octanoic acid, decanoic acid, ethyl acetate, ethyl butyrate, ethyl hexanoate, ethyl octanoate, ethyl decanoate, isobutyric acid, 2-methylbutyric acid, 3-methylbutyric acid, isovaleric acid, ethyl isobutyrate, ethyl 2-methylbutyrate, ethyl 3-methylbutyrate, ethyl isovalerate, carbonyl, lactone, diacetyl, 2,3-pentanedione, acetoine, γ-butyrolactone, ethyl lactate, diethyl succinate, Z-whiskylactone, E-whiskylactone, o and m-cresol, guaiacol, 4-methylguaiacol, eugenol, E-isoeugenol, 2,6-dimethoxyphenol, 4-allyl-2,6-dimethoxyphenol, vanillin, acetovanillone, propiovanillone, ethylvanillate, methylvanillate, furfural, 5-methylfurfural, 4-ethylphenol, 4-ethylguaiacol, 4-propylguaiacol, γ-lactones, γ-octalactone, γ-nonalactone, γ-decalactone, γ-undecalactone, γ-dodecalactone, 4-vinylphenol, 4-vinylguaiacol, or any combination of the foregoing.

In certain embodiments, the plurality of samples comprises a plurality of grapes. In certain embodiments, the plurality of samples comprises must or juice.

In certain embodiments, the plurality of samples comprises a grape product spiked with one or more compounds causing the one or more perceptual characteristics and/or a wine spiked with one or more compounds causing the one or more perceptual characteristics. In certain embodiments, the plurality of samples comprises a grape product spiked with a microbial organism. As an example, the microbial organism is Brettanomyces.

In certain embodiments, the optical signals from the samples comprise Raman spectra that were optionally obtained using a Surface Enhanced Raman Spectroscopy (SERS). In certain embodiments, the optical signals from the samples each comprise information from at least 10 wavelengths.

In certain embodiments, the training data further comprises information from an electronic sensor of volatile organic compounds. In certain embodiments, the training data further comprises information from infrared spectra, fluorescence, and/or visible spectra of the grape produce or the wine.

In some embodiments, training the feature extractor comprises training a variational autoencoder or a transformer model using the optical signals generated from the samples. In certain embodiments, the machine learning model is a neural network.

Any combination of the features mentioned for these aspects may be implemented together in methods of training a machine learning model in accordance with this disclosure.

Some aspects of this disclosure pertain to systems for predicting perceptual characteristics of wine. Such systems may be characterized by a processor and memory configured to: (a) receive an optical signal from a sample of a grape product or a wine, wherein the optical signal comprises a spectrum having characteristics influenced by chemical components of the grape product or the wine; (b) extract features from the optical signal by transforming information in the optical signal to a latent space, which latent space has reduced dimensions compared to dimensions of the optical signal; and (c) provide the features to a machine learning model that outputs (i) chemical composition information in the grape product or the wine, wherein the chemical composition information includes information about one or more compounds that are associated with one or more perceptual characteristics, and/or (ii) the one or more perceptual characteristics of a finished wine produced from the grape product or of the wine.

In certain embodiments, the one or more perceptual characteristics comprise a perceptual characteristic selected from the group consisting of a taste, an aroma, a mouthfeel, an appearance, and any combinations thereof. In certain embodiments, the one or more perceptual characteristics comprise a smoke taint perceptual characteristic. In certain embodiments, the one or more perceptual characteristics comprise a perceptual characteristic associated with one or more chemical byproducts of an organism, and wherein the chemical composition information comprises information about the one or more chemical byproducts of the organism. As an example, the organism may be Brettanomyces and the chemical composition information may be information about 4-ethylphenol, 4-ethylguaiacol, 4-ethylcatechol and/or 4-propylguaiacol.

In certain embodiments, the one or more perceptual characteristics comprise one or more metrics of the finished wine or a score representing the one or more metrics of the finished wine. In certain embodiments, the machine learning model is configured to output a recommendation of one or more wine making process parameters.

In certain embodiments, the system includes hardware in addition to a processor and memory. For example, the system may comprise a Raman spectrometer or an electronic sensor of volatile organic compounds.

In certain embodiments, the processor and memory are configured to extract the features by providing the optical signal to a variational autoencoder or a transformer model trained using training optical signals from training data from training samples of grape products and/or wines. In certain embodiments, the machine learning model was trained using training data comprising training mass spectrometry data obtained from training samples of grape products and/or wines.

In certain embodiments, the machine learning model is a neural network.

Any combination of the features mentioned for these aspects may be implemented together in systems of this disclosure.

Other aspects of this disclosure pertain to systems for training a machine learning model configured to predict perceptual characteristics of a wine. Such systems may be characterized by a processor and memory configured to:
receive training data for each of a plurality of samples, each sample comprising a grape product and/or a wine, wherein the training data for each sample comprises (a) an optical signal generated from the sample, and (b) (i) chemical composition information in the grape product, a finished wine produced from the grape product, or the wine, wherein the chemical composition information includes information about one or more compounds that are associated with one or more perceptual characteristics, and/or (ii) the one or more perceptual characteristics of the finished wine produced from the grape product or of the wine;
train a feature extractor using at least a portion of the training data, wherein the feature extractor is trained to extract features from the optical signals generated from the training samples by transforming information in the optical signals to a latent space, which latent space has reduced dimensions compared to dimensions of the optical signals; and
train a machine learning model, using at least a portion of the training data and at least features extracted from the training data by the feature extractor, wherein the machine learning model is trained to predict (i) the chemical composition information in the grape product, the finished wine produced from the grape product, or the wine, and/or (ii) the one or more perceptual characteristics of the finished wine produced from the grape product or of the wine.

In certain embodiments, the one or more perceptual characteristics comprise a perceptual characteristic selected from the group consisting of a taste, an aroma, a mouthfeel, an appearance, and any combinations thereof. In certain embodiments, the one or more perceptual characteristics comprise a smoke taint perceptual characteristic. In certain embodiments, the one or more perceptual characteristics comprise a Brettanomyces taint perceptual characteristic.

In certain embodiments, the chemical composition information comprises information about a catechin, tannin, anthocyanin, terpinol, linalool, geraniol, α-terpineol, citronelol, nerol, nor-isoprenoid, β-damsascenone, β-ionone, α-ionone, ethyl cinnamate, ethyl dihydrocinnamate, hexanol, Z-3-hexenol, E-2-hexenol, ethanol, fusel alcohol, isobutanol, 2 and 3-methylbutanol, isoamylalcohol, β-phenylethanol, methionol, fusel alcohol acetate, isobutyl acetate, isoamyl acetate, hexyl acetate, phenylethyl acetate, fatty acid, acetic acid, butyric acid, hexanoic acid, octanoic acid, decanoic acid, ethyl acetate, ethyl butyrate, ethyl hexanoate, ethyl octanoate, ethyl decanoate, isobutyric acid, 2-methylbutyric acid, 3-methylbutyric acid, isovaleric acid, ethyl isobutyrate, ethyl 2-methylbutyrate, ethyl 3-methylbutyrate, ethyl isovalerate, carbonyl, lactone, diacetyl, 2,3-pentanedione, acetoine, γ-butyrolactone, ethyl lactate, diethyl succinate, Z-whiskylactone, E-whiskylactone, o and m-cresol, guaiacol, 4-methylguaiacol, eugenol, E-isoeugenol, 2,6-dimethoxyphenol, 4-allyl-2,6-dimethoxyphenol, vanillin, acetovanillone, propiovanillone, ethylvanillate, methylvanillate, furfural, 5-methylfurfural, 4-ethylphenol, 4-ethylguaiacol, 4-propylguaiacol, γ-lactones, γ-octalactone, γ-nonalactone, γ-decalactone, γ-undecalactone, γ-dodecalactone, 4-vinylphenol, 4-vinylguaiacol, or any combination of the foregoing.

In certain embodiments, the plurality of samples comprises a grape product spiked with one or more compounds causing the one or more perceptual characteristics and/or a wine spiked with one or more compounds causing the one or more perceptual characteristics. In certain embodiments, the plurality of samples comprises a grape product spiked with a microbial organism.

In certain embodiments, the optical signals from the samples comprise Raman spectra. In certain embodiments, the optical signals from the samples each comprise information from at least 10 wavelengths. In certain embodiments, the training data further comprises information from infrared spectra, fluorescence, and/or visible spectra of the grape produce or the wine.

In some embodiments, the processor and memory are configured to train the feature extractor by training a variational autoencoder or a transformer model using the optical signals generated from the samples. In certain embodiments, the machine learning model is a neural network.

Any combination of the features mentioned for these aspects may be implemented together in systems of this disclosure.

These and other features of the disclosure will be presented below, sometimes with reference to drawings.

### BRIEF DESCRIPTION OF DRAWINGS

FIG. 1 schematically shows a process for predicting perceptual characteristics of a wine using Raman spectroscopy and machine learning.

FIG. 2 presents an example general structure of computational elements of a computational system that may be used to predict smoke taint or other perceptual characteristics in a finished wine.

FIG. 3A illustrates an example architecture and some functions of a variational autoencoder that may serve as a feature extractor of a model used to predict smoke taint or other perceptual characteristics in a finished wine.

FIG. 3B illustrates a simplified example architecture of a neural network that may serve as a perceptual characteristics machine learning model.

FIG. 4 illustrates a training method in which data and information are collected from and/or about one or more test or training samples. This data and information serve as one or more training sets for training a feature extractor.

FIG. 5 illustrates an example method for training a perceptual characteristics machine learning model. The training data includes data for a plurality of samples. For many or all samples, the training data includes one or more optical signals taken from the sample, and chemical composition information and/or sensory characteristics taken from the sample itself and/or a wine made from the sample.

FIG. 6 is a block diagram of an example of the computing device or system suitable for use in implementing some embodiments of the present disclosure. For example, device may be suitable for implementing some or all operations of determining smoke taint as disclosed herein.

FIG. 7 is graph showing Raman signal taken from synthetic wine samples spiked with o-guaiacol.

FIG. 8 is the graph of FIG. 7 but with background subtracted out.

FIG. 9 is a graph showing Raman signal taken from synthetic juice samples.

FIG. 10 is a graph showing Raman signal taken from wine samples.

FIG. 11 is a graph showing Raman signals that detected very low concentrations of guaiacol in synthetic juice.

FIG. 12 is a graph showing Raman signals that detected very low concentrations of guaiacol in synthetic wine.

### DETAILED DESCRIPTION

### Terminology

A “grape product” refers to any grape berry as well as any juice, must, concentrate or extract made from vinifera grapes, true or hybrid. It generally does not include alcoholic liquor.

The term encompasses grapes and any intermediate product in the wine making process, up to fermentation. Any of these materials can be used to produce an optical sample. The optical sample can provide input, directly or indirectly, to a machine learning model that predicts perceptual characteristics of finished wine.

The term “wine” refers to any alcoholic beverage or precursor of such beverage obtained, at least in part, by the fermentation of a sugar of a grape product. The term includes fortified wines as well as unfortified wines.

A “feature extractor” is a computational module configured to receive optical input data (e.g., optical spectra) from a sample and output a transformed representation of the data in a latent space, which typically has reduced dimensionality in comparison to the optical input data. The optical input data may be said to be projected onto the latent space. In some embodiments, a feature extractor is implemented as an autoencoder such as a variational autoencoder or an attention-based model such as a transformer model. Transformer models are widely described such as in A. Vaswani, et al., “Attention is all you need,” 31st Conference on Neural Information Processing Systems (NIPS 2017), Long Beach, CA, USA.

A “machine learning model” may be any trained computational model. In some embodiments herein, a machine learning model is configured to receive as inputs optical data reflective of chemical components in a grape material and/or a wine. Examples of machine learning models include neural networks, including recurrent neural networks, convolutional neural networks and generative adversarial networks, autoencoders, including variational autoencoders, random forests models, restricted Boltzmann machines, gradient boosted trees, linear regressions, support vector machines, attention-based models such as a transformer models, and probabilistic graphical models, including Bayesian networks. In some embodiments herein, machine learning models are trained using a training set that reflects a range of grape material samples (and finished wines) for which the model should be able to accurately predict perceptual characteristics such as smoke taint of a finished wine.

In general, though not necessarily, a neural network or autoencoder includes multiple layers. Each such layer may include multiple processing nodes, and the layers process in sequence, with nodes of layers closer to the model input layer processing before nodes of layers closer to the model output. In various embodiments, one layers feeds to the next, etc. The output layer may include one or more nodes configured to output information representing chemical composition and/or perceptual characteristics of a finished wine.

In some embodiments, the model has more than two (or more than three or more than four or more than five or more than six) layers of processing nodes that receive values from preceding layers (or as direct inputs) and that output values to succeeding layers (or the final output). Interior nodes are often “hidden” in the sense that their input and output values are not visible outside the model. In various embodiments, the operation of the hidden nodes need not be monitored or recorded during operation.

In some implementations, a machine learning model is trained using an unsupervised learning method. In some implementations, a machine learning model is trained using a supervised learning method. In some implementations, a machine learning model is trained using a semi-supervised learning method. For example, the training is performed using a set of training data, but only subset of that data has labels. In various implementations, generative adversarial networks, variational autoencoders, or other types of autoencoders are trained using semi-supervised learning methods.

In some embodiments, a machine learning model is trained to predict a smoke taint characteristic from data pertaining to optical data obtained from samples comprising a grape material and/or a wine. In some implementations, a smoke taint machine learning model is configured to receive as input features of the optical data extracted by, e.g., a feature extractor. The output of a smoke taint machine learning model may predict a chemical composition of wine and/or perceptual characteristics of wine.

The “perceptual characteristics” of wine refer to a multi-dimensional characterization of a finished wine's flavor and/or aroma. The multiple dimensions may be combined to provide a score of the finished wine. One of the multiple characteristics may be a smoke taint perceptual characteristic of the finished wine. Other perceptual characteristics may include taste (e.g., fruity, dry, bitter, sweet, acidic, salty, or savory), texture or mouthfeel (e.g., dry, smooth, astringent), after taste, aroma, bouquet, etc. The “smoke taint characteristic” of a wine is sometimes described as, e.g., “smokey”, “burnt” “ashy” or “medicinal.”

Optical analysis techniques include any technique that obtains optical signals from a sample. Such signals may be generated through interaction of the sample with a stimulus such as incident light. Samples may interact with stimulus to produce or modify light through, for example, absorption, emission, scattering, refraction, diffraction, etc. In some cases, an optical analysis technique contains information about vibrational characteristics of chemical bonds in chemical compounds within the sample. An example of an optical analysis technique that produces signals representing such vibrational characteristics is Raman spectroscopy.

Chemical composition information refers to qualitative and/or quantitative information about chemical compounds present in a sample. The information may uniquely identify specific chemical compounds such as m-cresol, syringol monoglucoside, and 4-methylguaiacol. The information may identify classes of chemical compounds such as phenols, glycosides, phenol glycosides, glucosides, methyl phenols, guaiacols, and syringols. The information may identify concentrations or amounts (absolute and/or relative) numerically.

Chemical composition information about a sample may be obtained directly or indirectly from samples. Examples of techniques for identifying chemical composition information include chromatography (including gas chromatography, liquid chromatography, and thin film chromatography), mass spectroscopy, absorption spectroscopy (including infrared (IR) spectroscopy, near infrared (NIR) spectroscopy, and visible spectroscopy), emission spectroscopy (including IR thermography and fluorescence spectroscopy), and electronic nose (e-nose). In some implementations, one or more of the following chemical analysis techniques are used to obtain chemical composition information: gas chromatography-mass spectroscopy (GC-MS), gas chromatography-tandem mass spectroscopy (GC-MS-MS), high performance liquid chromatography-mass spectroscopy (HPLC-MS), high performance liquid chromatography-tandem mass spectroscopy (HPLC-MS-MS), and high performance liquid chromatography-diode array detector-mass spectroscopy (HPLC-DAD-MS).

### Introduction and Context

Smoke taint is a phenomenon that wines made from grapes exposed to smoke having unpleasant flavor and aroma. The unpleasant flavor and aroma of smoke taint have been described in various ways, e.g., “wet ash tray”, “smokey”, “burnt,” “ashy,” “disinfectant” or “medicinal.”

Fire burning such as in wildfire burning or control burning produces volatile phenols including phenol, cresol isomers (o-, m-, p-), guaiacol, and syringol through thermal decomposition of lignin sources. They can enter into the grapevine via stomates in the leaves and are absorbed through the berry skin, followed by enzymatic conversion to phenolic glycosides. Wines made from grapes exposed to smoke contain elevated concentrations of volatile phenols and their glycosides which together cause unpleasant and undesirable sensory characters for both aroma and palate. In addition, the phenolic glycosides can release volatile phenols during winemaking and contribute to smoky flavors in wine. Due to the negative impact on a wine's sensory profile, the smoke-tainted grapes may be unsuitable for winemaking, causing substantial economic losses.

Many factors affect smoke taint, making it difficult to predict whether a specific batch of grapes would be unfit for making wine. For example, after smoke contacts grapes, volatile compounds such as volatile phenols contributing to smoke taint can penetrate grape skin and bond with sugars inside the grapes to form glycosides through glycosylation reactions. The volatile phenols may be reduced on the grape, making it difficult to predict smoke taint by smell or taste of the grapes.

When the grapes undergo fermentation, the acidity in the resulting wine begins to break the bonds between sugars and phenols, rendering the phenols volatile once again, causing smoke taint in the wine. This typically happens during fermentation but can continue to occur after the wine has been bottled or even when the wine is being consumed and catalyzed by enzymes in a consumer's mouth. Thus, wine making processes and conditions, even individual consumer physiology, may affect smoke taint.

Furthermore, the conditions of grapes and grapevines affect smoke taint. Factors such as differences in skin and cuticular waxes; berry physiology; distribution of glycosides between skin, juice and pulp; and the variation in the expression and activity of glycosyltransferase enzymes could affect uptake and metabolism of smoke-derived phenols. The growth stage, e.g., Eichhorn-Lorenz (E-L) stage of the grapevine affects smoke taint. Moreover, the location and condition of a fire affect whether grapes would be smoke tainted. Furthermore, different burnt plants create volatile phenols having various impacts on grapes and wines. Due to these and many other factors, it is difficult to predict whether a batch of grapes exposed to smoke would suffer smoke taint, leading to some wine experts to conclude that one can only determine smoke taint of a wine when one tastes it.

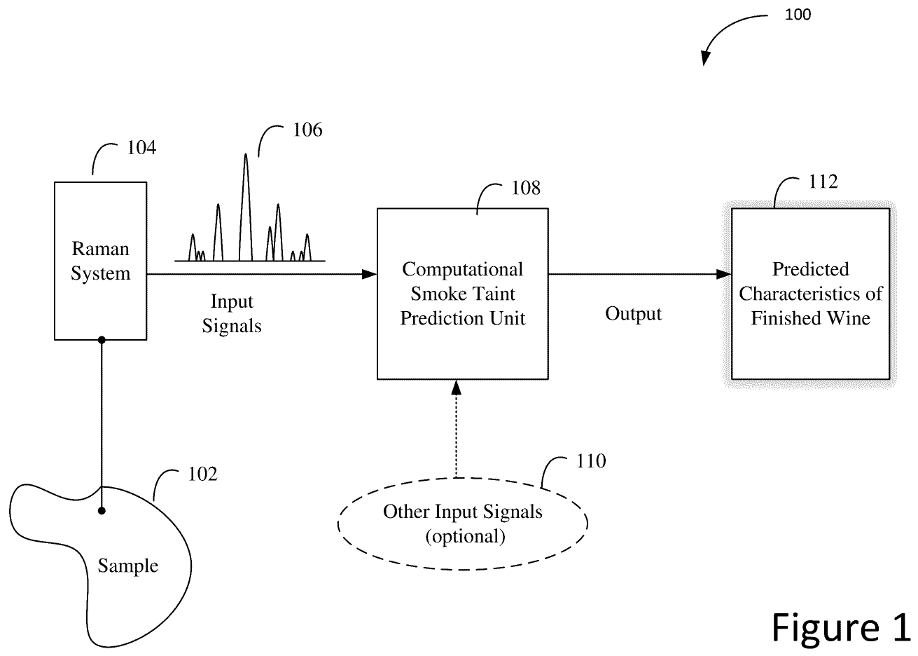

The instant disclosure provides technology that uses machine learning models to process optical signals obtained from grape products to predict smoke taint, providing fast and cost-efficient means for predicting whether wines made from grapes would be smoke tainted. FIG. 1 schematically shows a process 100 for predicting perceptual characteristics of a wine made according to some implementations using Raman spectroscopy and machine learning. A sample of grape product 102 such as grapes is prepared and analyzed by a Ramen spectroscopy system 102 to produce optical signals of Ramen spectra 106. The Raman spectra 106 is provided as model input to a computational smoke taint prediction unit 108. In some implementations, the computational smoke taint prediction unit 108 includes a feature extractor (e.g., a variational autoencoder or a transformer model) for extracting features in a latent space and a machine learning model that processes the extracted features to predict perceptual characteristics of the wine. In some implementations, optionally, other input signals 110 such as volatile compound signals of the grape product measured using an e-nose or suitable signals from the leaves of a grape plant or grapevine can be provided to the computational smoke taint prediction unit 108. After processing the Raman signal and optionally other input signals, the computational smoke taint prediction unit 108 provides as output its prediction of perceptual characteristics 112 of the wine.

While the disclosure focuses on models that predict smoke taint characteristics in finished wine, including predicting the presence of certain compounds or classes of compound that directly impact smoke taint characteristics, the disclosure is not limited predicting smoke taint characteristics. It extends to predicting any flavor characteristics or other perceptual characteristics of a finished wine. Therefore, the following description embodiments cover not just models (and model training techniques) for predicting smoke taint characteristics but for predicting finished wine flavor profiles and other perceptual characteristics.

For example, in some embodiments, a machine learning model is trained to predict a perceptual characteristic from data pertaining to optical data obtained from samples comprising a grape product and/or a wine. In some implementations, the perceptual characteristic may be associated with a byproduct of an organism. In some implementations, the perceptual characteristic may be a Brettanomyces taint. Brettanomyces produces phenols such as 4-ethylphenol, 4-ethylcatachol, and 4-ethylguaiacol that are known to cause or be associated with Brettanomyces taint perceptual characteristic, which is described as animal, leather, horse, smoky, ink, and medicinal.

In some implementations, a machine learning model is configured to receive as input features of the optical data extracted by, e.g., a feature extractor. The output of a machine learning model may predict a chemical composition of wine and/or a perceptual characteristic of wine caused by or associated with the chemical composition. For example, the output of a machine learning model may predict phenolics of the wine and/or an aroma and a mouthfeel caused by the phenolics.

For example, various phenolics in a grape product or a wine affect the taste and aroma of a wine. For example, tannins (including catechin, epicatechin, epicatechin-gallate, and epigallocatechin) cause a bitter taste. Ethyl isobutyrate, ethyl 2-methylbutyrate, ethyl 3-methylbutyrate (ethyl isovalerate), ethyl acetate, ethyl butyrate, ethyl hexanoate, ethyl octanoate, ethyl decanoate, contribute to fruity taste and aroma.

Various phenolics in a grape product or a wine also affects the mouthfeel of a wine. For example, tannins cause an astringent sensation in the mouth, contributing to “dryness” of the wine.

### Inputs to a Computational Smoke Taint Prediction Model

### Optical Analysis Techniques

In some embodiments, some or all input signals for a computational model are obtained using optical techniques for capturing chemical information in a sample.

In various embodiments, optical signals include values of optical intensity for light that has interacted with a sample. Such light may be reflected (e.g., as by specular reflection), scattered, diffracted, refracted, etc. by the sample. The optical intensity values may be provided as a function of light wavelength (e.g., for spectral data), light polarization state, and the like. In some implementations, light intensity may be polarization dependent, which polarization-dependent intensity may then be provided as a function of wavelength or other variables. The optical intensity values may be provided as a function of time.

In some embodiments, the optical signals provide vibrational information about chemical bonds of components of the grape product or wine. In some implementations, the optical signals provide rovibronic (rotational-vibrational-electronic) information of molecules in the sample. Examples of optical techniques that provide information of chemical bond vibrations include various techniques that employ infrared radiation and Raman spectroscopy.

In various embodiments, the optical signals used as inputs to a machine learning model are provided in the form of optical intensity value at each of multiple wavelength values. In some embodiments, optical intensity values are provided for at least about 3 wavelengths, or at least about 100 wavelengths, or at least about 1000 wavelengths. The number of such values may be a function of the instrument and its capabilities. For example, the number may be tied to the bandwidth of the instrument's detector and the wavelength range of the spectrometer.

Optical signals may be raw or substantially unprocessed signals from a spectrometer or other optical analysis tool. In some cases, optical signals are processed or modified prior to being provided to a machine learning model.

In certain embodiments, the optical techniques are nondestructive and rapid, at least by comparison to other analysis techniques such as chromatography and mass spectroscopy. In nondestructive techniques, the sample is not degraded or consumed by performing the optical analysis technique. For example, in some implementations using nondestructive and rapid techniques such as Raman spectroscopy, it takes 1-10 seconds per measurement, where 1-30 measurements are needed from each grape in a sample. In contrast, destructive techniques such as gas chromatography and mass spectroscopy may take 1 hour per measurement including sample preparation of a sample including, e.g., 1-20 grapes, 10-100 grapes, or a few hundred grapes.

In some embodiments, the optical analysis technique is one in which the optical signal from relevant organic compounds in the sample is not substantially obscured by signal from water. Because water will always be present in the sample, and typically is the dominant component of the sample, the optical technique employed to obtain data for input to the model should provide signal that is relatively undiluted or unobscured by signal from the water that is present. For this reason, infrared techniques such as Fourier-transform infrared (FTIR) and near IR spectroscopy are sometimes not employed. If they are employed, they may be used in conjunction with Raman spectroscopy. Raman spectra may be relatively immune from the obscuring effects of water.

In some embodiments, the optical signal is in the infrared, visible, and/or ultraviolet region(s) of the electromagnetic spectrum. In some embodiments, the optical signal is in the visible region of the electromagnetic spectrum.

In some embodiments, non-optical input signals may be provided together with optical input signals. As an example, signals indicating the presence or absence of certain volatile organic compounds or classes of such compounds may be provided. In many cases, such non-optical input signals are obtained rapidly (e.g., about 10 minutes or less) and nondestructively.

### Types of Samples

Most generally, the sample can be obtained at any stage of the winemaking process from the original plant material, typically grapes and/or leaves associate with the grapes, to the final wine produced by the process.

Examples of samples include the grapes themselves, prior to crushing, the must which includes a mixture of crushed grapes, including the skins, the seeds, the stems, and the juice prior to fermentation, partly or fully fermented juice, and the final wine itself. Must is a heterogenous mixture of solids and liquids, whereas juice is only a liquid. Juice is sometimes generated during the wine making process and may be fermented by itself or combined with must or other juices. In some embodiments, a sample is prepared by separating solids from must to provide liquid juice, which is used as the sample.

In some embodiments, a separate model is generated for each of these various types of sample. In some embodiments, a single model is suitable for two or more different types of sample (e.g., unprocessed grapes and must/juice).

In some embodiments, non-fruit components of a grape plant are used as samples or as complements to the fruit-based sample. These other sample may be used in addition to the grape-based sample or alone.

In some embodiments, grape leaves are used to generate signal. Grape leaves will sometimes provide stress responses to exposure to smoke or another stressor. Signatures of these stressors, which may include stress-induced chemical compounds, may serve as input to the model. For example, optical signals may be taken from samples derived from leaves of a grape plant.

### Preparation for Optical Analysis

A grape material or sample material may be prepared for optical analysis by any of various techniques. The preparation may vary based on the type of sample. For example, preparing a sample from the fruit itself may be different from preparing a sample from must, juice, or wine. In some implementations, a sample is acquired from a living plant using normal harvesting and/or fruit crushing techniques.

In some embodiments, samples are not prepared, or only minimally preparing, for optical analysis. For example, signals of grapes or plant leaves may be collected directly from a growing plant by using handheld devices. In some embodiments, a plant material is harvested from a vine or other plant and transported to a laboratory, where it is optically interrogated. In some embodiments, plant material is crushed in the laboratory to form samples.

In some embodiments, surface particulates or other potential contaminants on a grape skin or other solid sample are removed by using a surfactant.

In some embodiments, a plant sample is dried or otherwise treated to remove one or more chemical components. The resulting dried material may be optically interrogated.

In some embodiments, a sample is treated to enhance its optical signal. For example, a grape material may be crushed and spread onto the SERS substrate. Such treatments are described in further detail below.

### Raman Spectroscopy

In certain embodiments, Raman spectroscopy is performed on sample grape product. The resulting Raman spectrum may serve as the sole input or one of multiple inputs to a machine learning model.

Raman spectroscopy provides information derived from bonds of chemical compounds in a sample. The vibrational and rotational characteristics of these bonds, which may be detected in response to stimulation by a high-powered laser or other excitation radiation, provide an indication of which chemical compounds are present in a sample. A single chemical compound may have an identifiable discrete set of Raman peaks that may be relatively easy to identify.

However, when multiple chemical compounds are a sample, some of these peaks may overlap and provide an increasingly complex signal. For example, two different phenolic compounds might each contribute to a signature reflective of the presence of a phenyl group, but with subtle differences due to various different substituents on a phenol scaffold.

Raman spectra comprise intensity values as a function of wavelength lambda_x with an excitation at lambda_0. A Raman spectrum provides intensity values against (lambda_x−lambda_0). Even if lambda_0 is invisible or UV, lambda_x−lambda_0 may be in IR region. Laser wavelengths used in Raman spectroscopy may be in the ultra-violet through visible to, in some cases, near infra-red. Examples laser wavelengths include, but are not limited to, 244 nm, 257 nm, 325 nm, 364 nm, 457 nm, 473 nm, 488 nm, 514 nm, 532 nm, 633 nm, 638 nm, 660 nm, 785 nm, and 1064 nm.

Conventionally, Raman signatures are compared against the database of Raman spectra that represent many different types of samples containing mixtures of chemical compounds. Through this comparison, matching is conducted, and the Raman system provides an indicator of the likely composition of the sample under investigation. In the present invention, such comparison need not be undertaken. A computational model may provide the necessary analysis of Raman signals.

### Acquisition of Raman Spectra

In one approach, a grape or other grape product is simply exposed to a laser and the scattered light is captured across the relevant spectrum, as employed in conventional Raman spectroscopy. In other approaches, Raman spectroscopy is conducted in a manner that enhances the signal to noise ratio of the Raman signals. To this end, any one or more of various sample modifications may be employed.

### SERS

In some embodiments, surface enhanced Raman spectroscopy (SERS) is employed. In various implementations, the solid/semi-solid and liquid forms of grape products may be processed differently. For example, in SERS for solid or semi-solid samples, the grape product sample contacts or is disposed on a surface having nanoscale features, and through interaction of the excitation radiation, the nanoscale features and the sample compounds, together, provide the Raman signal. In liquid samples, the analyte is disposed on a nanostructured surface, or mixed with colloids with nano particles. The purpose of the sample preparation with SERS is to increase the interaction between samples and these nanoscale features. In SERS, the Raman signal is greatly enhanced. Without this approach, the Raman signal is extremely weak. The SERS enhancement factor can be as much as 10¹⁰ to 10¹¹.

SERS with grape product samples can be implemented in various ways. In a first approach, solid nanoparticles are provided with the sample. For example, nanoparticles may be sprinkled on wine grapes or mixed with must or juice. Some of the Raman signal is derived from compounds in the sample that are adsorbed on or otherwise adhered to surfaces of the nano particulate material. Highly stable Ag, Au, or Cu nanoparticles may be used in various implementations. Nanoparticles of various sizes and shapes may be used. For example, various implementations may use nanostars, nanotriangles, and nanospheres, flower-shaped Ag nanostructure (Ag-NF), Au nanodumbbells, unique branch shaped with sharp edges or tips, and snowflake-shaped gold nanoparticles (AuNPs).

In a second approach, the sample is disposed on a patterned substrate having nanoscale features thereon. A first type of substrate-based nanoscale feature is nanoparticles immobilized on solid substrates. One method of such a substrate uses a chemical tether to anchor Au/Ag NPs on a quartz surface. Another method uses thermo inject printer to disperse NPs on to paper or alumina filter. A sconed type of substrate-based nanoscale feature is nanostructures fabricated on solid substrates. Highly ordered metallic nanostructure arrays for SERS can be fabricated using nanolithography and related nanoimprint lithographic techniques, sometimes with subsequent deposition and etch to form favorable SERS enhancement. Metal film over nanosphere (FON), periodic nanoparticle arrays, or nanovoid arrays may be used, which are fabricated using nanosphere lithography. Table 1 shows how samples can be prepared to mix with nanoparticles in some implementations.

TABLE 1
Methods For Preparing Samples with Nanoparticles for SERS

| | | | | Attachment |
|---|---|---|---|---|
| Method | Source Material | Preparation | Nanoparticle | Method |
| 1 | Grape outer skin | No prep | Colloidal | Spray on |
| 2 | Grape outer skin | No prep/remove | Colloidal | Dip into and dry |
| | | cuticle | | |
| 3 | Grape skin only | skinned and | Colloidal | Direct mix |
| | | mashed | | |
| 4 | Grape skin only | skinned and | Solid | Spread thin layer |
| | | mashed | substrate | evenly |
| 5 | Grape skin only | skinned and | Colloidal | Direct mix |
| | | extracted for juice | | |
| 6 | Grape skin only | skinned and | Solid | Pipette & Drop |
| | | extracted for juice | substrate | juice |
| 7 | Grape inner skin | skinned and | Colloidal | Pipette & Drop |
| | | exposed | | colloids |
| 8 | Grape juice | crush and extracted | Colloidal | Direct mix |
| | (before/during/after | for juice | | |
| | fermentation) | | | |
| 9 | Grape juice | crush and extracted | Solid | Pipette & Drop |
| | (before/during/after | for juice | substrate | juice |
| | fermentation) | | | |

Some implementations use non-metallic nanostructure substrates. Substrates for Surface-Enhanced Raman Scattering Formed on Nanostructured Non-Metallic Materials: Preparation and Characterization by Krajczewski et al., Nanomaterials 2021, 11, 75 describes examples of non-metallic nanostructure substrates, which description is incorporated by reference.

Some implementations involve treatment of the semi-solid. The treatment involves skinning the grape, with or without the flesh, and then create a paste by thorough mixing, which can be performed by a blender for example. And then a sample of the mixture can be combined with nanoparticles to form a semi-solid paste for Raman spectroscopy. Alternatively, this paste can be spread on nanostructured substrates.

In a third approach, the sample is disposed on multiple different solid substrates, each having a different type of features disposed thereon to thereby provide different types of signal enhancement. In this third approach, multiple different spectra may be obtained for a given sample, one for each of the various substrates. Collectively, they are used as inputs to a machine learning model. Multiple different solid substrates may include different nanostructures (e.g., tubes, wires, cages, particles, etc.), each of optionally different size regimes. The multiple different solid substrates can be provided as continuous structure or a dispersed phase. In the former case, different regions of the structure comprise different nanostructures, and in the latter case, the different nanostructures form different colloidal suspensions with juice or other grape product.

A single sample such as single grape or single aliquot of grape must or juice may generate numerous data points. Each of these points may contain optical information such as a spectrum. In one example, an optical technique probes a single grape at multiple locations on the grape's surface. This process generates multiple optical signals such as multiple spectra from a single sample, a single grape in this example.

In some cases, a smoke taint analysis uses multiple samples, each of which may generate multiple inputs. Examples of the samples that can be used in a smoke taint analysis include multiple grapes from a single vineyard or from a single grape varietal within a single vineyard that was exposed to the same smoke conditions.

In some embodiments, a single spectrometer is configured to capture multiple sample signals at one time through multiplexing. In some examples, a single excitation beam from one light source may be split and directed onto multiple samples or multiple locations on a sample at one time. Then the scattered light or other optical signal from the samples may be collected by a single detector or by two or more detectors. In this manner, limited sampling resources can be used to quickly capture many data inputs.

In certain embodiments, using one or more light source and one or more detectors, 2 or more signals are captured concurrently, or 3 or more signals are captured concurrently, or 4 or more signals are captured concurrently, or 5 or more samples are captured concurrently, or 10 or more samples are captured concurrently.

In some implementations, time-based multiplexing may be employed. In such embodiments multiple channels having signals from multiple samples and/or from multiple locations on a sample are fed to a single detector. Through a switch or other device, signal from the different channels reaches the detector at different times. In some implementations, frequency-based detectors are employed, which optionally employ optical interference to provide spectral information about the sample. As examples, such systems may employ FTIR spectroscopy.

In some embodiments, one or more detectors is configured to determine signal intensity as a function of wavelength (e.g., the detectors directly generate spectral information). For example, the detector may employ a diffraction grating. In some embodiments, one or more detectors are not configured to directly discriminate between signal intensity at different wavelengths (e.g., the detectors are non-spectral detectors).

### Non-Raman and Non-Grape Inputs to a Model

Sometimes a Raman signal is not the only input to a model. Rather, a complementary input signal is used along with Raman signals. Such signals may be optical and/or non-optical. In some embodiments, such signals are obtained from a plant producing the sample or even from the sample itself. In some embodiments, such signals are obtained rapidly and/or non-destructively.

One example of a non-Raman input is signal a direct measurement that indicates the presence (and optionally the amount) of one or more volatile organic compounds or classes of volatile organic compounds. Such information may be obtained using an electronic sensor of organic compounds. Such sensors may employ semiconductor devices and a sensing material such as a biological material. In some embodiments, volatile organic compound signals are obtained using electronic nose (e-nose) technology. Many e-nose sensors detect volatile organic compounds. Electronic noses generally comprise gas sensors (e.g., metal oxide semiconductors) and a pattern recognition system that recognizes simple and complex odors and compounds associated with the odors. Various available e-noses have different sensor types, data processing, pattern recognition systems, and/or functioning principles. Various e-nose products are commercially available. For example, a portable e-nose developed by the Digital Agriculture, Food and Wine (DAFW) Group includes an array of nine gas sensor with sensitivity to different gases. The nine sensors and the gases they are sensitive to are: (i) MQ3=ethanol; (ii) MQ4=methane; (iii) MQ7=carbon monoxide; (iv) MQ8=hydrogen; (v) MQ135=ammonia, alcohol, and benzene; (vi) MQ136=hydrogen sulphide; (vii)=MQ137=ammonia; (viii) MQ138=benzene, alcohol, and ammonia; and (ix) MG811=carbon dioxide. In some implementations, nanostructured substrate, particularly lithographically printed substrates, may be used to measure adsorbed volatile organic compounds by sensors, in conjunction with optical spectroscopy.

In some embodiments, the optical input data from grape material or finished wine is supplemented with optical or other data from non-grape parts of a grape plant. For example, the additional data may come from leaves of a grape plant.

Similar to grape berries, grapevine leaves can also take up guaiacol and other volatile phenols and transform them into glycosides. For example, glycosyltransferase enzymes have been found in Gewurztraminer leaves which are able to convert guaiacol, 4-methylguaiacol, syringol, 4-methylsyringol, m-cresol and o-cresol into glycosides. In contrast to grapes, leaves have a large surface area available for adsorption and take up of volatile phenols from smoke, even very early in the season. Therefore, in some implementations, grapevine leaves are processed and analyzed in similar ways as grape berries to generate optical and/or chemical data. Then relevant features or signatures from leaves are extracted and provided to a machine learning model for predicting perceptual characteristics of wine including smoke taint.

Some implementations use chemical composition systems such as GC-MS or HPLC to extract from grapevine leaf samples chemical signatures relating to, e.g., guaiacol, cresol, phenol, syringol, 4-methylguaiacol, 4-methylsyringol, monoglucosides (MGs), gentiobiosides (GGs), pentosylglucosides (PGs), rutinoside, etc. Some implementations use optical techniques such as Raman spectroscopy to obtain optical data from grapevine leaves, then extract optical features, which features may be related to chemical signatures.

In some embodiments, the optical input data is supplemented with other information about the wine or wine-making process. Such information may include one or more details about the process used to produce the must or juice (e.g., how long crushed grape juice remains in contact with skins and stems, the filtering techniques, etc.), details about the fermentation process (e.g., yeast type, temperature, and pH control), and/or details about the barrel aging process.

When non-Raman signals are used, they may be provided as inputs along with Raman signal to a feature extractor or other input side of a smoke taint detection computational model. In some embodiments, Raman and non-Raman signals are provided to different portions of a computational model. For example, Raman signals may be provided to a feature extractor, while non-Raman signals are not provided to a feature extractor but are, instead, provided directly to a smoke taint machine learning model.

In certain embodiments, a method of predicting perceptual characteristics employs a microbiological technique to identify the presence, and optionally the amount, of a microbe such as Brettanomyces in a grape material sample. Such technique may be used alone or in combination with information derived from an optical signal processed as described herein to predict the chemical composition and/or the perceptual characteristics of a finished wine produced from the grape material. The microbiological technique may also be employed to monitor the amount or change in amount of the microbe (e.g., Brettanomyces) in a grape material as it undergoes processing to form a finished wine. Again, such monitoring may be used alone or in conjunction with information derived from an optical signal processed as described herein.

The microbiological technique may include microscopic imaging, culturing, nucleic acid sequence detection and the like. Examples of nucleic acid sequence detection include polymerase chain reaction (PCR) techniques, such as quantitative PCR (qPCR), and whole genome sequencing such next generation sequencing (NGD).

An example process for detecting the presence and/or likely impact of Brettanomyces on a wine's perceptual characteristics includes some or all of the following operations:

Collect a sample: Use a sterile syringe or pipette to collect a small sample to test. Conduct this in a clean environment to avoid contamination.

Culture the sample: Add the sample to a suitable growth medium that contains the necessary nutrients for growing Brettanomyces. For example, the medium may be a Lysine medium or Wallerstein laboratory nutrient (WLN) agar. Incubate the medium at a suitable temperature (around 25° C.) and monitor it for the growth of Brettanomyces. The time required for growth can vary depending on the initial concentration of Brettanomyces in the sample.

Confirm the presence of Brettanomyces: Once the growth of Brettanomyces has been observed, one can confirm its presence by performing various tests. These tests may include microscopy, PCR analysis, biochemical tests, a sensory test, or any combination thereof.

Optionally conduct a Raman spectroscopy analysis of Brettanomyces in the sample or the sample chemical components: One can collect the cells from the cultured medium and prepare them on a glass slide for Raman analysis.

In some embodiments, a microbiological technique for identify the presence, and optionally the amount, of Brettanomyces in a grape material sample is used in conjunction with an analytical method for determining the presence or amount of any one or more compounds typically produced by Brettanomyces such as 4-ethylphenol, 4-ethylguaiacol, and 4-ethylcatechol. Such compounds may be identified by Raman spectroscopy, gas chromatography-mass spectrometry (GC-MS), liquid chromatography-mass spectrometry (LC-MS), and the like.

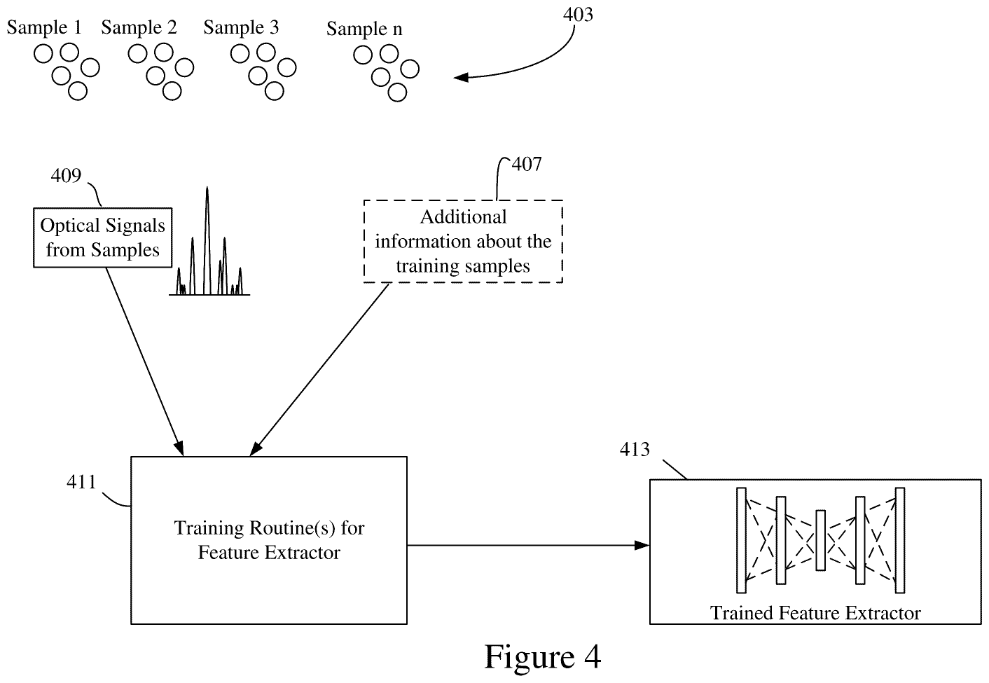

In some embodiments, information obtained from a microbiological technique for identifying the presence or amount of Brettanomyces may be provided as an input, optionally with optical and/or chemical information from a grape material sample to a machine learning model such as described herein. For example, the “other input signals” 110 shown in FIG. 1 may comprise information from the microbiological technique. Similarly, the “additional information about the training samples” 407 shown in FIG. 4 may comprise information from the microbiological technique.

In some embodiments, a machine learning model using information obtained from a microbiological technique for identifying the presence or amount of Brettanomyces employs feature extraction as described herein.

In some implementations, samples are tested for Brettanomyces at various stages in the wine-making process. As an example, consider the following opportunities for testing for Brettanomyces, optionally along with other components that influence a wine's perceptual characteristics.

Grapes: Testing the grapes for the presence of Brettanomyces before they are harvested can help to identify potential sources of contamination and enable proactive measures to be taken to minimize the risk of Brettanomyces growth.

Fermentation: Monitoring the fermentation process for any changes in aroma or flavor can help to detect the presence of Brettanomyces early on. It is also important to ensure that proper sanitation practices are followed to minimize the risk of contamination.

MLF (malolactic fermentation): Monitoring the MLF process for any changes in aroma or flavor can help to detect the presence of Brettanomyces. Brettanomyces can thrive in environments with low pH and high alcohol, so adjusting the pH and managing the alcohol levels may help to control its growth.

Barrel and tank: Sampling the wine in barrels and tanks for the presence of Brettanomyces can detect contamination at this stage of the process. Sanitation practices may be followed of adjusted to minimize the risk of contaminating future wine batches in the barrel or tank.

Bottling: Testing the wine for the presence of Brettanomyces before it is bottled can help to ensure that the final product is free from contamination. Sanitation practices may be followed of adjusted to minimize the risk of contaminating future wine batches in the bottling process.

In some embodiments, any two or more of these testing, monitoring, or sampling operations are performed. In some cases, using the output of the machine learning model at the multiple stages of the wine making process can account for potential variations in the presence or concentration of Brettanomyces during the wine making process, thereby allowing targeted treatment to manage and/or prevent Brettanomyces spoilage. It should be understood that the multi-stage sampling and machine learning analysis described here can be extended to detect or monitor the presence, amount, or impact on perceptual characteristics of any other (non-Brettanomyces) component of wine such as any of the various phenols or classes of phenols described herein.

### Output of a Computational Wine Characteristics Prediction Model

In some embodiments, a model is trained to predict one or more perceptual characteristics of wine that will be produced from the tested material or of wine that has already been produced. In some cases, the perceptual characteristics may correspond to characteristics that would be described by a wine expert such as a sommelier or by a trained wine consumer. These perceptual characteristics may take the form of a vector or multi feature, multi-metric description. In some embodiments, a model is trained to predict one or more chemical components of wine that will be produced from the tested material. Such chemical components may include one or more compounds known to impact smoke taint.

### Perceptual Characteristics

Examples of some dimensions of the perceptual characteristics include mouthfeel (astringency, dryness, viscosity), sweetness, bitterness, acidity, savory, aroma, oak, fruit (including red/dark fruit or tropical fruit), earth, mineral, chalk, vegetable, grassy, sulfur, floral, petrol, body, and finish.

In some implementations, the perceptual characteristics of a wine include an appearance of the wine such as colors and clarity.

In many embodiments, one or more dimensions of a wine's perceptual characteristic represent the contribution of smoke taint. The unpleasant flavor and aroma of smoke taint have been described in various ways, e.g., “wet ash tray”, “smokey”, “burnt,” “ashy,” “disinfectant”, “tarry” or “medicinal.”

In some implementations, a perceptual characteristic of a wine represents a perception associated with a byproduct of an organism in a grape product or a wine. In some implementations, the organism is Brettanomyces and the perceptual characteristic of a wine represents the perception of Brettanomyces taint, which is described as animal, leather, horse, smoky, ink, and medicinal. When Brettanomyces grows in wine it produces several compounds including 4-ethylphenol, 4-ethylguaiacol, 4-ethylcatechol, and 4-propylguaiacol, which cause Brettanomyces taint perceptual characteristic and can alter the palate and bouquet of the wine. At low levels some winemakers agree that the presence of these compounds has a positive effect on wine, contributing to complexity, and giving an aged character to some young red wines. However, when the levels of the sensory compounds greatly exceed the sensory threshold, their perception is almost always negative. Wines that have been contaminated with Brettanomyces taints are often referred to as “Bretty”, “metallic”, or as having “Brett character.”

In some implementations, perceptual characteristics of a wine include a perceptual characteristic indicating whether or not consumers favorably perceives the finished wine produced from the grape product or the wine. In other words, whether the consumers like the wine. Such a characteristic may include a binary score indicating whether the consumer likes the wine given multiple characteristics (e.g., taste, aroma, mouthfeel, appearance) of the wine.

In some implementations, perceptual characteristics of a wine include a favorability score indicating how favorably consumers consider the finished wine produced from the grape product or the wine. In other words, how much the consumers like the wine on a scale of, e.g., 0-5, 1-5, 1-10, 1-100, 80-100, given multiple characteristics of the wine.

In some implementations, the perceptual characteristics of a wine include a favorability score indicating in certain consumption context how favorably consumers consider the finished wine produced from the grape product or the wine. In some implementations, the consumption context is a pairing of the wine with various food items (e.g., seafood, white meat, red meat, dessert). In some implementations, the consumption context is a time of the consumption, such as a time of the day or season of a year.

In some implementations, the machine learning model predicts multiple perceptual characteristics that form a flavor profile or perceptual profile of a grape product or wine. Such a flavor profile or perceptual profile may be used as a target profile in some applications to guide a wine making process or flavor engineering as further described hereinafter.

In various implementations, Machine learning models providing these perceptual characteristics as output are trained by supervised training methods using data labeled with these perceptual characteristics.

### Chemical Characteristics

In certain embodiments, a smoke taint machine learning model outputs information that predicts the presence and/or quantity of particular chemical compounds (or features of compounds) in a sample or in a finished wine made from grapes that produced the sample. Among the compounds or features are one or more that may impart smoke taint in a finished wine. The output may be in the form of a vector or matrix of chemical compounds (and optionally quantities of these compounds) present in the sample or wine. In various embodiments, at least some of the compounds in the vector or matrix may impact perception of the finished wine.

In various implementations, the information of the chemical compounds provided as output by the machine learning model corresponds to chemical composition data measured from training samples using chemical analysis techniques described herein, e.g., GC-MS, GC-MS-MS, HPLC-MS, HPLC-MS-MS, and HPLC-DAD-MS. The chemical compounds are associated with smoke taint or other perceptual characteristics of grape products or wines. After training, the machine learning model receives as input test data of optical data and provides as output (or a prediction) chemical composition information corresponding to data measured by mass spectrometry in some implementations. In other words, the model uses optical data to generate data similar to mass spectrometry data. Because optical data can be easier, cheaper, and faster to obtain than mass spectrometry data, some implementations provide easier, cheaper, and faster means to determine chemical composition and perceptual characteristics than conventional, mass spectrometry methods.

Considering multiple possible compounds, rather than only one or a small number of such compounds, may facilitate detecting smoke taint because certain compounds associated with smoke taint only impart a smoke taint perceptual characteristic to the finished wine and not to the grape or juice used to make the wine. Further, in some cases, a compound that can cause smoke taint does so only in combinations with certain other compounds. For example, two wines may contain the same smoke taint imparting compound, but the smoke taint perception only exists in one of the wines. This may be because one of the wines lacks certain compounds that mask the taint or meld the taint with other perceptual characteristics. Thus, in two different samples containing the same amount and type of smoke taint-producing compounds, one sample may exhibit a degradation in flavor due to smoke taint and another may not exhibit such degradation.

A smoke taint machine learning model may provide chemical information in various ways. In the case of a direct representation, the model's output may identify particular chemical compounds. Numerical values associated with the various chemical compounds may represent relative or absolute concentrations or other quantitative information about individual chemical compounds in the wine sample.

In cases where the output does not directly correspond to particular chemical compounds and/or their relative quantities in a sample or in wine made from the sample, the output may represent more abstract information about the chemical composition. For example, the output may contain information about combinations of compounds or about moieties that produce common or overlapping vibrational signal characteristics. In some cases, the output provides information about classes of chemical compounds.

Examples of classes of compounds that may be represented as such in output from a smoke taint model include alcohols, phenolic compounds (e.g., phenolic acids, polyphenols, condensed tannins, flavanols, flavonols, and stilbenoids), anthocyanins, amines, esters such as acetates, butyrates, and hexanoates, carboxylic acids such as isovaleric acids and octanoic acids, thiols, and glycosides of any of these. In some cases, the output of the model specifies the presence of one or more phenols or classes of phenol. Examples of classes of phenol include catechins, tannins, anthocyanins, quercetins, guaiacols, cresols, and syringols. Within these classes are derivates of the phenols including glycoside of the phenols. Some of these compounds such as guaiacols, cresols, and syringols are characteristic of smoke impacted grapes, but whether they impart smoke taint characteristics to a finished wine is dependent upon the presence and/or relative amounts of other components commonly found in wine, many of which are phenolic compounds such as tannins and flavanols. Examples of such other components that impact smoke taint characteristics include catechins, tannins, anthocyanins, including polymeric anthocyanins, quercetins, including quercetin glycosides, and any combination of these.

In some implementations, a machine learning model provides as output perceptual characteristics other than smoke taint, or compounds contributing to these other perceptual characteristics. For example, in some implementations a machine learning model provide as output perceptual characteristics including sweetness, bitterness, acidity, fruit, aroma, mouthfeel, colors.

The machine learning model may also provide as output compounds contributing to these perceptual characteristics. For example, the compounds include sugars and glycosides contributing to sweetness, acids contributing to acidity, tannins (including catechin, epicatechin, epicatechin-gallate, and epigallocatechin, etc.) contributing to bitterness, tannins contributing to astringency and dryness, phenolics contributing to aromas and colors.

Phenolics and other compounds affecting the aroma of wines include but are not limited to the following categories.

Grape origins: terpenols (linalool, geraniol, α-terpineol, citronelol, nerol), nor-isoprenoids (β-damsascenone, β-ionone, α-ionone), ethyl cinnamate, ethyl dihydrocinnamate, hexanol, Z-3-hexenol, E-2-hexenol.

Fermentative origin: ethanol, fusel alcohols (isobutanol, 2 and 3-methylbutanol, isoamylalcohol, β-phenylethanol, methionol), fusel alcohol acetates (isobutyl acetate, isoamyl acetate, hexyl acetate, phenylethyl acetate), fatty acids and their ethyl esters (acetic acid, butyric acid, hexanoic acid, octanoic acid, decanoic acid, ethyl acetate, ethyl butyrate, ethyl hexanoate, ethyl octanoate, ethyl decanoate), branched acids and their ethyl esters (isobutyric acid, 2-methylbutyric acid, 3-methylbutyric acid, isovaleric acid, ethyl isobutyrate, ethyl 2-methylbutyrate, ethyl 3-methylbutyrate, ethyl isovalerate), carbonyls and lactones (diacetyl, 2,3-pentanedione, acetoine, γ-butyrolactone), ethyl esters of major acids (ethyl lactate, diethyl succinate).

Wood origin: lactones (Z-whiskylactone, E-whiskylactone), volatile phenols (o and m-cresol, guaiacol, 4-methylguaiacol, eugenol, E-isoeugenol, 2,6-dimethoxyphenol, 4-allyl-2,6-dimethoxyphenol), vanillins (vanillin, acetovanillone, propiovanillone, ethylvanillate, methylvanillate), furfural, 5-methylfurfural.

Brettanomyces phenols: 4-ethylphenol, 4-ethylguaiacol, 4-ethylcatechol, 4-propylguaiacol.

Miscellaneous: γ-lactones, γ-octalactone, γ-nonalactone, γ-decalactone, γ-undecalactone, γ-dodecalactone, 4-vinylphenol, 4-vinylguaiacol.

For further descriptions of these and other aroma related phenolics and other compounds, see, Red Wine Technology, Chapter 20 (Antonio Morata, 2018), which is incorporated by reference by its entirety.

Phenolics affecting appearance of wines include, e.g., anthocyanins, pyroanthocyanins, vitisins, portisins, and oxovitisins. Examples of moieties include aromatic components (particularly aryl), ether groups, ester groups, alcohol groups, carboxylic acid groups, and combinations of any of these in a single compound or in multiple compounds of a sample.

In some examples, the chemical information output by a smoke taint model contains information about bond types of one or more compounds present a sample or in a finished wine. Examples of such bond type information includes C—H (aromatic C—H bond bend, stretch, or out of plane deformation), O—H (hydroxyl group stretch), C—O (pyran ring stretch), C—OH (stretch of phenols), C—O—H (deformation of phenols), C═O (ester stretch), S—H (thiols), and C—C═C (aromatic ring stretch).

Examples of specific chemical compounds that may be identified in the output of the model include cinnamic acid, resveratrol, hydroxycinnamic acid, caffeic acid, gallic acid, rutin, hexanoic acid-ethyl ester, octanoic acid-ethyl ester, nonanoic acid-ethyl ester, ethyl 9-decenoate, decanoic acid-ethyl ester, dodecanoic acid-ethyl ester, octanoic acid-3-methylbutyl ester, alpha-methyl-benzene methanol, guaiacol, 4-methylguaiacol, syringol, 4-methylsyringol, o-cresol, m-cresol, p-cresol, and glycosides of any of these.

Examples of compounds directly associated with smoke taint and that may be identified in the output of the model include m-cresol, o-cresol, p-cresol, 4-methylguaiacol, guaiacol, syringol, 4-methylsyringol, phenol, and glycosides of any of these. Examples of other compounds that can be associated with smoke taint and that may be identified in the output of a model include thiols such as benzylthiol (also called benzyl mercaptan), benzenethiol (also called thiophenol), 2-methoxybenzenethiol, 4-methylbenzenethiol, 3-methylbenzenethiol, and 2-methylbenzenethiol. Examples of glycoside classes include monosaccharides, disaccharides, and trisaccharides. Examples of specific types of glycosides include monoglucosides, pentosylglucosides, gentiobiosides (GGs), rutinosides, and the like. Some examples glycosides include syringol gentiobioside, methylsyringol gentiobiosides (e.g., 4-methyl syringol gentiobioside), monoglucosides (MGs) of methylguaiacol and methylsyringol, guaiacol rutinoside, 4-methyl guaiacol rutinoside, and cresol rutinosides (e.g., p-cresol rutinoside).

### Profiles, Scores, and Grades

In some embodiments, the direct or indirect output of a machine learning model is or contains raw information about a chemical profile or matrix of a finished wine produced from the sample. In such cases, it may be up to a consumer of this information to determine whether smoke taint, Brettanomyces, or other perceptive characteristic is likely to be present in the finished wine. In other embodiments, a machine learning model as described herein is configured to output a high-resolution score that correlates with the likelihood that the finished wine product will experience smoke taint, Brettanomyces influence, or another defined perceptive characteristic. In still other embodiments, a machine learning model as described herein is configured to output a grade of a finished wine. In some cases, the grade is a binary result that simply indicates whether the finished wine will likely suffer from smoke taint, be influenced by Brettanomyces, or have some other defined perceptual characteristic. Note that a score, grade, or other model output metric may be scalar or multi-dimensional. For example, a score or grade may comprise multiple metrics based on perceived characteristics

### Computational Systems

In some cases, the model is simply a single neural network or other form of machine learning model that takes optical data (e.g., Raman spectra) as input and generates predicted chemical compositions and/or perceptual characteristics of the final wine product as outputs.

However, this may be challenging because optical data such as Raman spectra are often represented by so many variables in a very large multidimensional space. In various embodiments, Raman spectra used as input to a machine learning model comprise at least about 3 wavelengths, or at least about 100 wavelengths. Often, input from a single sample includes over 1000 dimensions of information. Therefore, in some embodiments, to reduce the computational complexity, the system or model is designed in such a way that it projects the raw Raman multidimensional input onto a much lower dimensional, latent space.

In one approach, a model first transforms or converts the Raman or other optical signal to a reduced dimensional representation. In various embodiments, the latent space representation remains abstract, without directly identifying chemical compounds or classes of such compounds. The process of transforming or converting the optical signal is sometimes referred to as feature extraction.

Features extracted from a feature extractor may be provided as input to a smoke taint machine learning model that is trained to predict perceptual characteristics and/or compounds present in a finished wine, such as a wine made from a grape material sample.

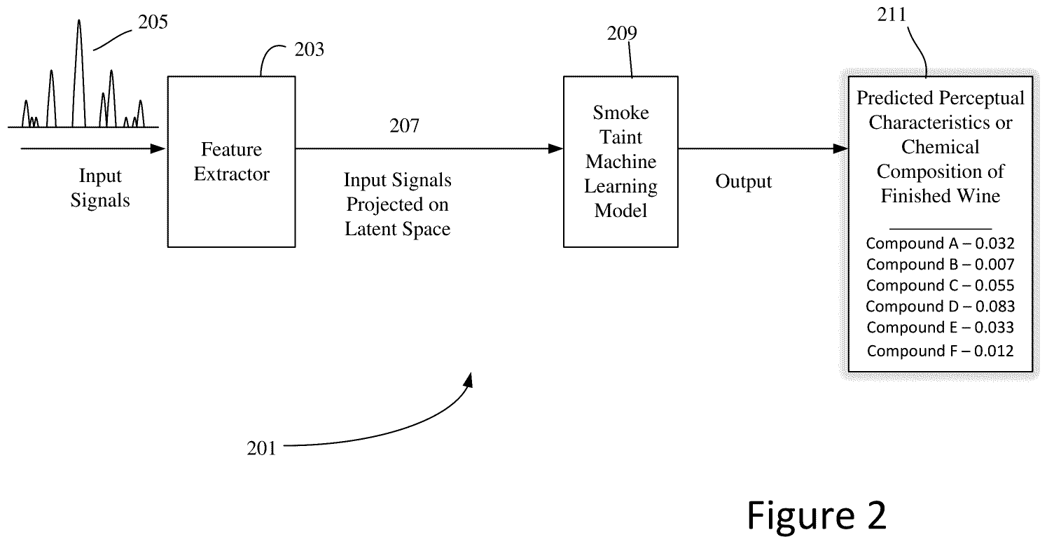

FIG. 2 presents an example general structure of computational elements of a computational system 201 that may be used to predict smoke taint or other perceptual characteristics in a finished wine. In the depicted embodiment, a feature extractor 203 is configured to receive optical input signals 205 such as Raman spectra taken from one or more samples. Feature extractor 203 is configured to project information from signals 205 onto a latent space and thereby produce features 207 (input signals projected onto latent space). A smoke taint machine learning model 209 is configured to receive as inputs features 207 and output predicted perceptual characteristics and/or information about the chemical composition of a finished wine. See 211.

In some embodiments, a computational system employs three processing units. These include:
1. Preprocessor configured to preprocess a sample's spectral data to improves its usefulness for a feature extractor and/or a smoke taint machine learning model. In some embodiments, a preprocessor is configured to reduce noise in the optical data, normalize the optical data across multiple samples, reduce instrument error, and/or otherwise improve the optical data.
2. Feature extractor configured to transforms spectral data (optionally preprocessed) into reduced dimensional latent space. This may be accomplished using, e.g., a variational autoencoder trained on optical data or principal component analysis.
3. Smoke taint machine learning model trained to receive optical input data (e.g., features extracted from spectral data) and predict an organic compound profile and/or perceptual characteristics of finished wine.

### Preprocessing

Various preprocessing techniques may be used to preprocess a sample's spectral data to improve subsequent modeling in some implementations. One category of preprocessing is scatter-correction methods, including Multiplicative Scatter Correction (MSC), Inverse MSC, Extended MSC (EMSC), Extended Inverse MSC, de-trending, Standard Normal Variate (SNV) and normalization.

Another category of preprocessing of spectral data involves spectral derivatives, which include Norris-Williams (NW) derivatives and Savitzky-Golay (SG) polynomial derivative filters. Both methods use a smoothing of the spectra prior to calculating the derivative in order to decrease the detrimental effect on the signal-to-noise ratio that conventional finite-difference derivatives would have. Review of the Most Common Pre-processing Techniques for Near-infrared Spectra (Rinnan, et al., Trends in Analytical Chemistry, Vol. 28, No. 10, 2009) describes methods of scatter correction and spectral derivative, which description is incorporated by reference.

In addition, in some implementations, spectra data undergo feature extraction by wavelength window, within which a multilayer perceptron (MLP) or a deep learning neural network or a polynomial feature extraction is used to capture the spectral feature.

Moreover, some implementations use certain pre-generated spectra where a convolution operation by wavelength window is performed.

### Feature Extractor

A feature extractor may make data from samples of grape materials or wine more useful for further analysis. A feature extractor may be configured to identify features in optical signal data that are particularly relevant to a wine's perceptual characteristics such as smoke taint. In some cases, a feature extractor reduces noise in the optical signals obtained from samples. Examples of noise that may be reduced by a feature extractor include signal arising from sources of signal that can interfere with the signal created by the chemical components relevant to smoke taint. Examples of other sources of interfering signal may include instrument error, such as that related to positioning, alignment, and/or calibration of the optical system.

The output of a feature extractor—which may be referred to as features of the input optical data—may be suitable as an input for another application such as a smoke taint model that can predict smoke taint characteristics of a finished wine.

Various data processing routines or structures may be employed as feature extractors. One of these is an autoencoder such as a variational autoencoder that transforms or projects input optical data onto a latent space.

### Inputs to a Feature Extractor

In certain embodiments, the input to a feature extractor is raw or minimally pre-processed optical data from samples based on grape materials or wine. For a given sample, the input data may comprise many broadband spectra, with one spectrum for each of multiple readings taken from a sample. The spectrum from any reading from a sample may be represented as optical intensity data at any number of wavelengths.

In some embodiments, feature extractor is trained to receive input data in addition to optical signals from samples. Examples of such other information includes electronic sensor signals (e.g., eNose signals) or features extracted from NIR or FTIR from the sample and/or finished wine, information about exposure of grape plants to smoke such as exposure time, location, duration, etc., grape plant information (e.g., the vineyard location, terroir, vine stock, the grape varietal, etc.), information about the wine making process, and the like.

Moreover, other input data for the feature extractor include but are not limited to: (1) time between exposure to smoke and grape harvest, (2) duration of smoke exposure, (3) intensity of smoke exposure, (4) grape maturity stage during exposure to smoke, and (5) grape variety.

In some implementations, wine making process information may be used as input data. Such information about the wine making process includes, e.g., harvest time, crushing conditions, must handling, time between crushing and fermentation, temperature prior to, during or after fermentation or incubation, incubation period, yeast, inoculum size, additives, pH, and substrate concentration.

### Outputs From a Feature Extractor

In certain embodiments, the output of the feature extractor is a de-noised version of the input optical input signals.

In certain embodiments, the output of the feature extractor is a latent space representation of the optical input signals. The latent space may be provided by transforming the data space of the raw optical data (e.g., intensity as a function of wavelength) to an abstract representation of the data that does not directly map to physical dimensions such as wavelength, intensity, etc. In certain embodiments, the latent space is determined using a machine learning model such as a neural network, a transformer model, or an autoencoder. In certain embodiments, the latent space is determined using principal component analysis.

In certain embodiments, the output optionally includes information about the sample such as information about its vineyard (e.g., the location of the vineyard), details of its known exposure to smoke, information about the grape varietal(s) in the sample, information about the wine making process(es) employed to make the finished wine, and the like.

### Feature Extractor Characteristics

As indicated, a feature extractor may be configured to receive spectra and optionally other information about the test samples such as information about origin and processing of grape material. In certain embodiments, the outputs are or include an abstract representation of the input optical signals in a latent space. In certain embodiments, the output is an encoded version of the input data, which output is provided from an encoder portion of an autoencoder.

In certain embodiments, the feature extractor is a multilayer model having the form of a neural network or an autoencoder such as a variational autoencoder. In some embodiments, the feature extractor is configured to project input data to a latent space in a probabilistic manner. For example, the feature extractor may project data not as discrete values but as distributions of values on axes in latent space. The distributions may be characterized by, e.g., their central tendencies (means, medians, mode, etc.) and/or their variances in the latent space. Some variational autoencoders process input data in this manner. In certain embodiments, the feature extractor has at least five layers. In certain embodiments, the feature extractor has at least 5 nodes. In certain embodiments, a feature extractor contains about 3 to 20 layers. In certain embodiments, the input layer of the feature extractor has at least 5 nodes.

In certain embodiments, the feature extractor has a convolutional layer. In some cases, the convolutional layer is configured to filter wavelength and/or photon energy information. The convolution layer may be configured to extract relevant features from the input data such as multi-wavelength characteristics of the input data.

In various embodiments, a feature extractor defines a latent space representation of the optical input signals. The latent space is a multidimensional space having a reduced number of dimensions in comparison to the input data, e.g., the spectra from grape material and/or wine test samples. The data space of the raw input data (e.g., intensity as a function of wavelength) is understandable in terms of the physical reality of the metrology. For example, the raw data has dimensions corresponding to various wavelengths of the input spectra. By contrast, the latent space representation is an inherently abstract representation of the input data and is not easily understandable in terms of the underlying physical dimensions such as wavelength, intensity, polarization, etc. Nevertheless, the latent space representation of the data may embed the information content from the input spectra and/or other input signals. And the physical construction of the spectra may be decoded by an appropriately trained decoder. Without using a decoder, it may be difficult or impossible to discern what the physical contributions are to the data contained in the latent dimensional space. In certain embodiments, the latent space is determined using a machine learning model such as a neural network, a transformer model, or a variational autoencoder.

In certain embodiments, the feature extractor outputs sample information presented in the latent space of a variational autoencoder, a transformer model, or another machine learning model. In some implementations, the data in the latent dimensional space serves as an input into a different machine learning model, one that can predict perceptual characteristics of a finished wine such as smoke taint.

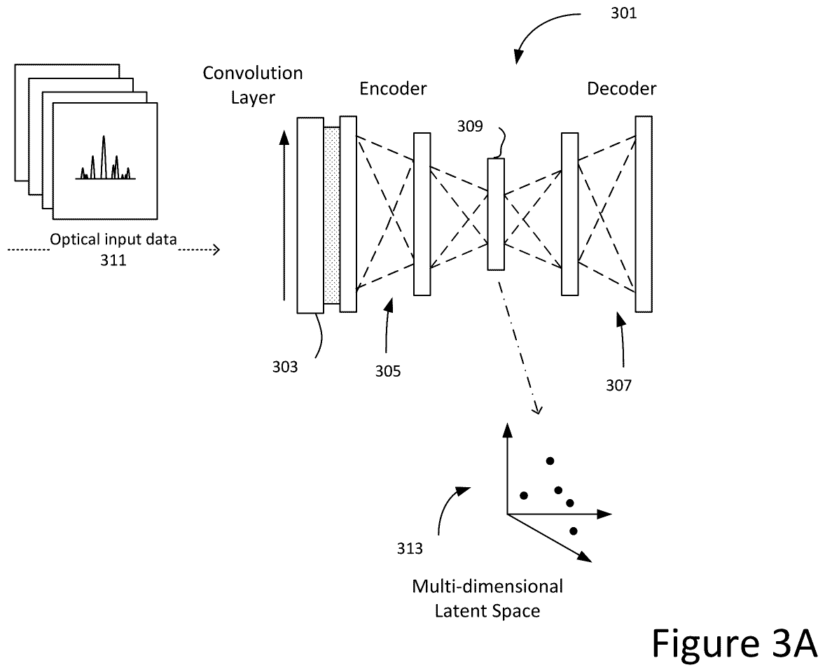

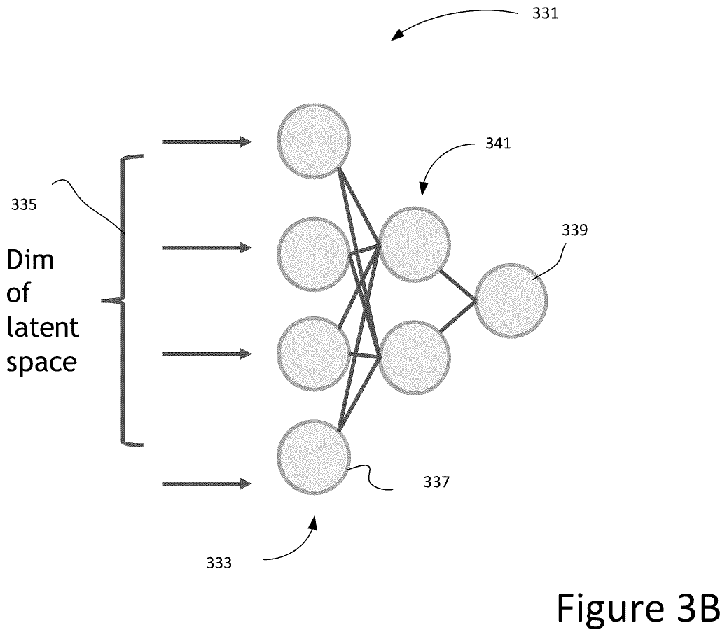

FIG. 3A illustrates an example architecture and some functions of a variational autoencoder that may serve as a feature extractor as described in this section. As illustrated, a variational autoencoder 301 optionally includes a convolution layer 303 at an input side, a multilayer encoder portion 305, a multilayer decoder portion 307, and a hidden or latent space portion 309. Variational autoencoder 301 is configured to receive input optical data 311 acquired from a test sample. The input data 311 may be provided in the form spectra obtained from various positions on a grape surface. Optionally, the optical input data is organized and provided to the convolution layer 303 that is configured to extract potentially relevant features from the spectra. Variational autoencoder 301 is configured such that the input data filtered by convolution layer 303 is processed by the encoder layers 305 and decoded by the decoder layers 307. Between the encoder and decoder portions is a hidden layer 309 configured to hold the fully encoded metrology data in a latent space.

In the depicted embodiment, the hidden or latent space portion 309 holds a multi-dimensional latent space representation 313 of the fully encoded data. The latent space representation 313 comprises multiple data points, each associated with a particular sample or a particular optical reading taken from a sample.

Latent space 313 includes points corresponding to multiple optical spectra taken from a test sample. For example, the latent space points may correspond to input spectra taken for ten grapes at five locations on each grape (producing fifty points total). In some embodiments, the latent space points may be statistically analyzed to provide one or more pieces of information about the points collectively. For example, the points may be analyzed to provide a central tendency (e.g., their mean or median in the latent space), a variation, and/or other collective representation in latent space. The resulting collective information may be used as input to a machine learning model trained to predict smoke taint and/or other perceptual characteristics of a finished wine.

Note that standard techniques for interpreting Raman signals such as determining Raman peak positions (typically provided as wave number values), peak amplitude, and peak width may miss subtle but important characteristics of a Raman signal such as small substructures within Raman peaks or distant peaks that correlate with a peak of interest under certain conditions. Such characteristics may be dictated by the chemical environment of compounds of interest. Computational analysis using machine learning as described herein can address this issue by, for example, extracting features representing the difficult to observe subtle characteristics.

Also, in some cases, the resolution of a Raman tool used to analyze grape material samples may be limited compared to that of much more expensive laboratory-grade Raman tools. As such important features of a Raman signal may be obscured or not immediately apparent, at least to a human analyst. Computational analysis using machine learning as described herein can address this issue by, for example, extracting features not apparent to human observers but that represent these missing characteristics.

In some embodiments, a machine learning model as described herein can utilize peak shift information in a Raman signal that might otherwise be difficult to interpret. Changes in the position of Raman bands can indicate changes in the chemical composition of the sample. For example, the peak of aromatic benzene rings at 1599 cm⁻¹, which is associated with the C═C stretch, can shift in the presence of smoke taint compounds such as guaiacol or syringol.

### Machine Learning Model

In various embodiments, a smoke taint model is configured to receive features extracted from optical data of a test sample and output either (a) chemical composition information about the sample or a finished wine made from the sample, or (b) perceptual information about the finished wine. Such model may be configured in many ways.

Examples of machine learning model types that may be used for the smoke taint machine learning model include neural networks, including recurrent neural networks and convolutional neural networks, autoencoders, including variational autoencoders, self-attention-based models such as a transformer models, random forests models, restricted Boltzmann machines, gradient boosted trees, linear regressions, support vector machines, and probabilistic graphical models, including Bayesian networks. Input nodes may include those for each of multiple latent dimensions in a trained feature extractor for processing optical signals from grape material samples, and, optionally, one or more other types of input data. When designed as a neural network, for example, the smoke taint model may have three or more layers.

FIG. 3B illustrates a simplified example architecture of a neural network 331 that may serve as a smoke taint machine learning model. As shown, model 331 includes an input layer 333 that has nodes 335 configured to receive latent dimension data representing extracted features from optical data of a test sample.

In the depicted embodiment, input layer 333 also has a node 337 configured to receive other data. Examples of such other information includes electronic sensor signals (e.g., eNose signals) from the sample and/or finished wine, information about exposure of grape plants to smoke such as exposure time, location, duration, etc., grape plant information (e.g., the vineyard location, terroir, vine stock, the grape varietal, etc.), information about the wine making process, and the like.

Model 331 also has an output layer 339 and one or more hidden layers 341 (only one is shown in FIG. 3B). Output layer 339 comprises one or more nodes configured to present one or more perceptual characteristics of finished wine (e.g., smoke taint characteristics) and/or chemical composition information about the test sample or a finished wine made from the sample.

### Applicability of Machine Learning Models to Wines

Some implementations train and apply different machine learning models for different grape varieties. Although the uptake and metabolism of volatile phenols are generally similar between grape varieties, there are meaningful differences in volatile phenol glycoside profile between varieties or cultivars. For example, Chardonnay grapes were observed to accumulate higher concentrations of phenol pentosylglucosides (PhPGs), cresol pentosylglucosides (CrPGs) and cresol rutinosides (CrRGs) but lower levels of guaiacol gentiobiosides (GuGGs) than Merlot grapes when both varieties were exposed to smoke for the same duration under controlled conditions. In Shiraz berries have higher levels of guaiacol glycosides, including both MG and disaccharides, compared with Chardonnay berries after smoke exposure. Similarly, smoke-exposed Cabernet Sauvignon and Pinot Noir grapes had lower levels of guaiacol glycosides than Chardonnay and Sauvignon Blanc. Also, because white wines are made without using grape skins, stems, or seeds as red wines are, the effects of chemical compositions of white wine grapes on smoke taint tend to be weaker than red wine grapes. Therefore, in some implementations, white wine grapes and red wine grapes are processed by different models. In some implementations, different varieties, variants of a variety, or groups of varieties are processed by different models. For example, in some implementations, pinot noir grapes and cabernet grapes may be processed by two different models. In some implementations, pinot noir grapes and cabernet grapes may be grouped together and be processed by one model. In some implementations, two different variants of pinot noir grapes are processed by two different models.

### Training of the Computational System

Training data from training samples is used to train a computational system such as one employing a feature extractor and a smoke taint machine learning model. The training samples may include grape material and/or wine. In some embodiments, training data includes at least the following information obtained from multiple training samples: (1) optical data, (2) chemical data, and, optionally, (3) perceptual characteristics of finished made from the sample (or which is the sample). In some embodiments, the training data includes only the optical and chemical data.

As an example, a grape product (e.g., grapes, must, juice, or finished wine) is analyzed by an optical technique such as Raman spectroscopy to produce Raman spectra. The same grape product is analyzed by a chemical technique such as GC-MS to produce a representation of information about organic compounds in the sample. In some implementations, one or more of the following mass spectrometry techniques may be used to quantify organic compounds in the sample: gas chromatography-mass spectroscopy (GC-MS), gas chromatography-tandem mass spectroscopy (GC-MS-MS), high performance liquid chromatography-mass spectroscopy (HPLC-MS), high performance liquid chromatography-tandem mass spectroscopy (HPLC-MS-MS), high performance liquid chromatography-diode array detector-mass spectroscopy (HPLC-DAD-MS). In some implementations, other chemical analysis techniques may be used to quantify organic compounds in the sample.

When preparing a grape material sample for gas chromatography (GC), solid phase microextraction (SPME) may be employed. In some implementations, SPME is followed by GC-MS or GC-FID. SPME is a sample preparation technique that involves the extraction and concentration of volatile and semi-volatile compounds from a sample onto a coated fiber, which is then desorbed into the gas chromatography system for separation and detection. This technique can be used in combination with GC-MS or GC-FID for the quantitative analysis of flavor compounds in complex matrices.

The same grape product is also converted to a finished wine, which is evaluated to provide chemical and/or perceptual characteristics of the wine. Thus, the training set may include data triplets, each generated from a single sample and each including optical data (e.g., Raman spectra), chemical compound information (e.g., GC-MS, GC-MS-MS, HPLC-MS, HPLC-MS-MS, HPLC-DAD-MS data), and perceptual characteristics of the finished wine. In some cases, multiple optical signals are taken from the same sample. For example, Raman spectra are acquired at multiple locations on a single grape.

For each sample of grape material, the perceptual information may be in the form of (a) expert classifications, and/or (b) chemical composition. The chemical composition may indicate the presence and amount of certain chemicals implicated in smoke taint such as guaiacols, syringols, and cresols. The chemical composition may be provided in a form that maps to, e.g., GC-MS representations of the chemical composition.

In some embodiments, training samples include different groups of grapes, which are measured, fermented and converted to wine. Some of the grapes have been exposed to smoke and other have not. Different grape samples may be exposed different amounts of smoke, from different fires, etc. In some examples, each training sample has a set of Raman spectra collected from its grapes, optionally from different surface positions of each grape, and each training sample also has perceptual characteristics and/or chemical composition information of finished wine produced from the corresponding grapes. Thus, the training set includes pairs of Raman spectra from grapes and chemical composition information and/or perceptual characteristics of wine produced from the same grapes.

In some implementations, multiple spectra are generated for each sample (e.g., each grape or each group of similar grapes), and these multiple spectra are paired with a single chemical measurement or a single perceptual test result. Once trained, however, a machine learning model will not require optical measurements having a large sample size (e.g., not as large as required for a technique such as GC-MS).

In some embodiments, to increase the range of training data, a sample or a wine made from a sample is spiked with one or more compounds that are known to impact wine flavor or other perceptual characteristic. Examples of such compounds include cresols, guaiacols, syringols, and/or other compounds implicated in smoke taint.

In some cases, no additional chemical testing need be made on the spiked sample because the parent sample may have a known chemical composition and the material spiked is provided in a known amount. With this information, the complete chemical composition of the training sample may be known.

In some embodiments, a sample is spiked with a microbe such as Brettanomyces. In some implementations, the microbe is cultured or otherwise treated under conditions encountered in wine making and allowed to produce its compounds that may influence the perceptual characteristics of the finished wine.

The following examples of grape materials and wines may be used together as in training samples:
Wine having smoke taint perceptual characteristics
Wine without smoke taint perceptual characteristics
Spiked wine that before spiking might not have smoke taint perceptual characteristics (e.g., DOE generated spiking)
Grape material that produces a wine having smoke taint perceptual characteristics
Grape material that produces a wine without smoke taint perceptual characteristics
Spiked grape material that before spiking might not produce a wine having smoke taint perceptual characteristics (e.g., DOE generated spiking)
Time since or duration of smoke exposure
Grape at different maturity stage
Duration and intensity of smoke exposure
Variety of smokes from different vegetation plants
Multiple varietals of any of the above
Multiple vineyards producing any of the above
Multiple levels of known smoke exposure for any of the above
Multiple different wine making processes employed for any of the above

As examples, training may be accomplished using any combination of the above information.

A few examples of training data combinations include:
Raman spectra from grapes, chemical compound information from the grapes, and chemical and/or perceptual characteristics of wine produced from the grapes
Raman spectra from grapes, chemical compound information from must or juice, and chemical and/or perceptual characteristics of wine produced from the must or juice
Raman spectra from must or juice, chemical compound information from must or juice, and chemical and/or perceptual characteristics of wine produced from the must or juice
Raman spectra from finished wine, chemical compound information from the finished wine, and perceptual characteristics of the finished wine

Other types of training data may be employed. For example, optical and/or chemical information from non-grape portions of a grape plant may be employed. An example of the non-grape portion is a leaf or leaves from the plant.

Moreover, other training data may include but are not limited to: (1) time between exposure to smoke and grape harvest, (2) duration of smoke exposure, (3) intensity of smoke exposure, (4) grape maturity stage during exposure to smoke, and (5) grape variety.

In some implementations, wine making process information may be used as training data. Such information about the wine making process includes, e.g., harvest time, crushing conditions, must handling, time between crushing and fermentation, temperature prior to, during or after fermentation or incubation, incubation period, yeast, inoculum size, additives, pH, and substrate concentration.

In certain embodiments, a machine learning model is trained in stages, particularly in embodiments that convert optical spectra input to features in a reduced dimensional space. In some embodiments, training takes place in these two primary stages:

Train a feature extractor, which may be implemented as a variational autoencoder, a transformer model, or similar system, using only the spectra acquired from the training samples. This training may be unsupervised, semi-supervised, or self-supervised. It identifies relevant patterns in the spectral data and defines the latent space.

Train a smoke taint machine learning model, which may be implemented as a neural network or similar system, using (a) the transformed data from the latent space and (b) chemical and/or perception data about the finished wine product. This may be a supervised training process, with the information about the finished wine product serving as labels.

Thus, a first stage of the training may involve training a feature extractor to extract features from optical input signals, which features are relevant to the true information content across many samples.

In the second stage of training, features produced during the first stage are related to chemical and/or perceptual characteristics of the final wine product. This training may involve training a machine learning model to predict chemical and/or perceptual characteristics of a finished wine. In some embodiments, the chemical characteristics may be provided as a list or matrix representing information about chemical compounds in the finished wine. In some embodiments, perceptual characteristics are generated by trained wine reviewers or other experts.

In some embodiments, the first stage will generate a feature extractor by training a variational autoencoder, which identifies a latent space to which spectra were projected into during unsupervised or semi-supervised training. The second stage keeps the encoder portion of the variational autoencoder from the 1^{st} phase, between spectra (input layer) and latent space, and uses latent space representations of the spectra and maps the latent space to chemical and/or sensory parameters.

In some cases, the labels used in supervised training of the machine learning model (second stage) are chemical compositions of the samples that produce the Raman spectra and/or finished wines made from such samples. The chemical compositions may be obtained from any of various techniques. For example, they may employ GC-MS data derived from the sample. GC-MS data indicate the presence or absence of various compounds and their relative amounts. In some embodiments, other analytical techniques are employed that likewise produce information about individual chemical compounds in the training samples.

In some implementations, HPLC, infrared (IR) spectroscopy, near infrared (NIR) spectroscopy, FT-IR spectroscopy, fluorescence spectroscopy, visible spectroscopy, UV-visible spectroscopy, IR thermography, inductively coupled plasma optical emission spectrometer, hyperspectral camera, nuclear magnetic resonance (NMR) spectroscopy, cyclic voltammetry, electronic nose (e-nose), or any combination of these methods is used to determine chemical composition information of samples. When any such technique is employed in training a machine learning model, such technique may also be used to generate data from a test sample, which data may be used as an input of the machine learning model during assessment of test samples. For example, a machine learning model may be configured to receive both Raman spectra and fluorescence information from a test sample.

In wine assessment, fluorescence spectroscopy may be used for various purposes. For example, it may be used to analyze phenolic compounds. Phenolic compounds, including flavonoids and non-flavonoids, play a role in wine quality, affecting its color, taste, and mouthfeel. Fluorescence spectroscopy may be employed to determine whether these compounds are present, as many of them are naturally fluorescent.

Further, fluorescence spectroscopy may be used to detect adulterants and contaminants. Fluorescence spectroscopy can help identify the presence of adulterants or contaminants in wine, such as the addition of water or other substances that may alter the wine's quality or authenticity. Further, fluorescence spectroscopy may be used to monitor wine aging. Fluorescence spectroscopy can be used to monitor the changes in wine composition during aging, as some compounds may exhibit altered fluorescence properties as they oxidize or undergo other chemical transformations. Still further, fluorescence spectroscopy may be used to assess microbial spoilage. Some microorganisms responsible for wine spoilage, such as Brettanomyces, can produce fluorescent compounds as metabolic byproducts. Fluorescence spectroscopy can be used to detect these compounds and assess the presence and extent of microbial spoilage in wine.

### Training a Feature Extractor

FIG. 4 illustrates a training method in which data and information are collected from and/or about one or more test or training samples. This data and information serve as one or more training sets for training a feature extractor. In the depicted embodiment, multiple training samples 403 provide training optical signals 409 and optionally training additional information 407 about the samples.

The training information and data from all these, and optionally other, sources are provided as training data to machine learning training routine(s) 411, which uses these inputs to train a machine learning model 413, typically in unsupervised self-supervised, or semi-supervised fashion.

In some implementations, the training routine(s) 411 obtains supervisory signals from the training data itself, such as by leveraging the underlying structure in the data. This is process is referred to by some data scientists as self-supervised learning. The general technique of self-supervised learning is used to predict any unobserved or hidden part (or property) of the optical input signals from any observed or unhidden part of the input.

In certain embodiments, a feature extractor is trained in a semi-supervised fashion that employs both labeled and unlabeled training data. Examples of semi-supervised training techniques are described in Yang, X., Song, Z., King, I., & Xu, Z. (2021). A Survey on Deep Semi-supervised Learning. http://arxiv.org/abs/2103.00550, which is incorporated herein by reference in its entirety. In some embodiments, a feature extractor is trained in one or more iterations, and in fact may employ multiple separate machine learning models, some serving as a basis for transfer learning of later developed refinements or versions of the feature extractor. In some embodiments, a feature extractor is partially trained using supervised learning and partially trained using unsupervised learning.

In some embodiments, learning is conducted in multiple stages using multiple training data sources via a mechanism such as transfer learning. Transfer learning is a training process that starts with a previously trained model and adopts that model's architecture and current parameter values (e.g., previously trained weights and biases) but then changes the model's parameter values to reflect a new or different training data. In various embodiments, the original model's architecture, including convolutional windows, if any, and optionally its hyperparameters, remain fixed through the process of further training such as via transfer learning.

In certain embodiments, one or more training routines produce a first trained preliminary machine learning model. Once fully trained with training data, the preliminary model may be used as a starting point for, e.g., training a second machine learning model. The training of the second model starts by using a model having the architecture and parameter settings of the first trained model but refines the parameter settings by incorporating information from additional training data. The second model may be better able to extract features relevant to smoke taint in a finished wine.

In certain embodiments, training the feature extractor is conducted in a manner that trains a variational autoencoder. To this end, the training may employ loss functions and/or other techniques that train the feature extractor to project input data to a latent space in a probabilistic manner. In some implementations, the loss functions may employ a regularization term utilizing Kullback-Leibler divergence. The feature extractor projects data not as discrete values but as distributions of values on axes in latent space. The distributions may be characterized by, e.g., their central tendencies (means, medians, etc.) and/or their variances in the latent space. The training may encourage the learned distribution (in latent space) to be similar to the true prior distribution (the input data).

### Training a Smoke Taint Machine Learning Model

Training a smoke taint machine learning model may be performed in various ways. In general, a smoke taint machine learning model may be trained using supervised or semi-supervised training. As mentioned, examples of semi-supervised training techniques are described in Yang, X., Song, Z., King, I., & Xu, Z. (2021). A Survey on Deep Semi-supervised Learning. http://arxiv.org/abs/2103.00550, previously incorporated herein by reference in its entirety.

In some implementations, the training is performed using supervised learning in which chemical composition information (including compounds known to impart smoke taint) and/or perceptual characteristics of finished wines are used as labels. Training is conducted in a way that relates features of optical signals from grape material samples to the labels. In some embodiments, a training matrix is employed that includes, for individual samples, concentration or other information about multiple chemical compounds in a sample or a wine made from the sample.

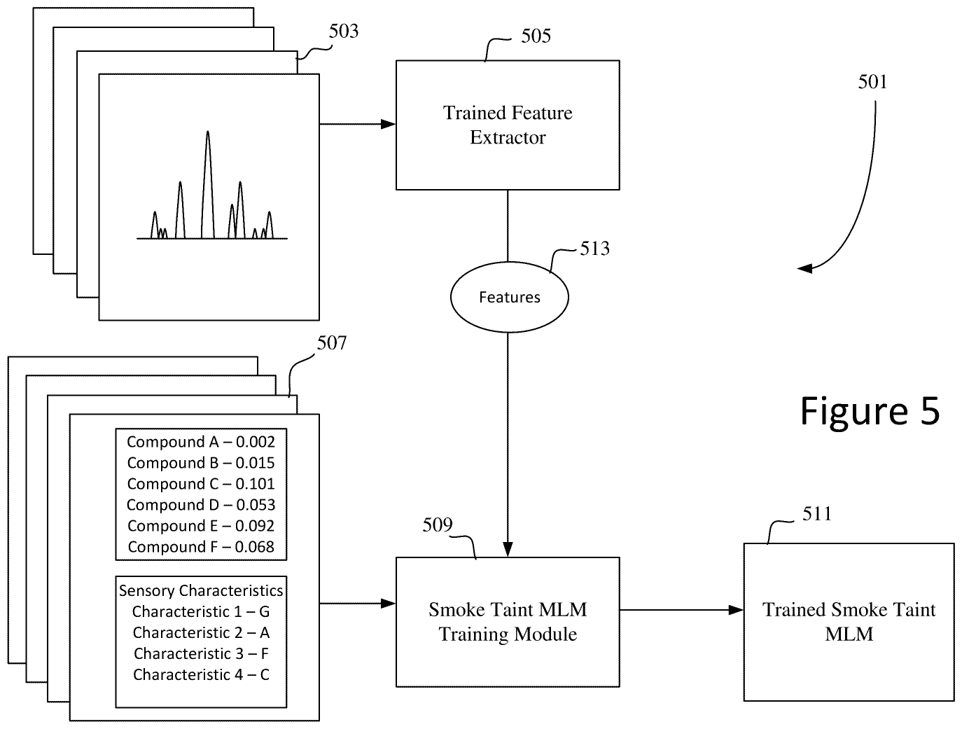

FIG. 5 illustrates an example method 501 for training a smoke taint machine learning model. The training data includes data for a plurality of samples. For many or all samples, the training data includes one or more optical signals taken from the sample, (see e.g., spectra 503) and chemical composition information and/or sensory characteristics (e.g., 507) taken from the sample itself and/or a wine made from the sample. In embodiments in which the trained machine learning model employs inputs in addition to optical signals from samples, such other information is included in the training data. Examples of such other information includes electronic sensor signals (e.g., eNose signals) from the sample and/or finished wine, information about exposure of grape plants to smoke such as exposure time, location, duration, etc., grape plant information (e.g., the vineyard location, terroir, vine stock, the grape varietal, etc.), information about the wine making process, and the like.

The optical signals and, optionally, other information are provided to a previously trained feature extractor 505. In some embodiments, feature extractor 505 was trained as described elsewhere herein. In some examples, feature extractor 505 is implemented as a variational autoencoder or a transformer model. Feature extractor 505 extracts features 513 from the optical signals of training data.

Features 513 along with chemical composition information and/or sensory characteristics 507, and, optionally, other types of training data are provided to a smoke taint machine learning model training module 509, which is configured to a train a smoke taint machine learning model 511 via conventional training procedure.

In certain embodiments, a smoke taint machine learning model is trained using a cost function such as a mean squared error (MSE) function for multivariate training data. This approach uses a sum of weighted error between the model prediction and actual labels of the training data over multiple dimensions. The multivariate information may include multiple pieces of information about the finished wine and/or the sample, notably chemical composition information and/or perceptual characteristics.

### Process Flows for Training and Applying Computational Systems

A general process flow may include some or all the operations listed in the following sequence:
1. collect sample [e.g., grape product and optionally other plant material that was possibly exposed to smoke]
2. optionally treat sample [e.g., wash grapes; e.g., apply grape product to SERS surface]
3. make multiple measurements of sample to generate a set of spectra for the sample [e.g., choose twenty grapes and take readings at five distinct locations on each grape]
4. preprocess the sample's multiple spectral data to, e.g., reduce noise, normalize data across multiple samples, reduce instrument error, etc.
5. transform preprocessed spectra into points in a reduced dimensional latent space via, e.g., an autoencoder
6. determine characteristics of the transformed spectra in the latent space [e.g., determine a centroid or other statistical features of the transformed data]
7. predict organic compound profile and/or perceptual perception of finished wine from the transformed spectral data via a trained machine learning model
8. decide how to treat grape product based on prediction from operation 7

### Training—Example 1

Train an autoencoder or similar system using only optical spectra acquired from the grape product training samples. This training is unsupervised, semi-supervised, or self-supervised. It identifies relevant patterns in the grape product spectral data and defines the latent space.

Train a neural network or similar system using (a) the transformed grape product data from the latent space and (b) chemical and/or perception data about the finished wine product. This is a supervised training process, with the information about the finished wine product serving as labels.

### Inference Scenario 1 (Based on Example 1 Training)

1. collect grape product sample that was possibly exposed to smoke
2. prepare sample [e.g., wash grapes; e.g., apply grape product to SERS surface]
3. make multiple measurements of sample to generate a set of spectra for the sample [e.g., choose twenty grapes and take readings at five distinct locations on each grape]
4. transform preprocessed spectra into latent variables in a reduced dimensional latent space via, e.g., the autoencoder previously trained
5. predict organic compound profile of finished wine from the latent variables via the previously trained machine learning model
6. decide how to treat grape product based on prediction from operation 5

### Inference Scenario 2 (Based on Example 1 Training)

1. collect grape product sample that was possibly exposed to smoke
2. prepare sample [e.g., wash grapes; e.g., apply grape product to SERS surface]
3. make multiple measurements of sample to generate a set of spectra for the sample [e.g., choose twenty grapes and take readings at five distinct locations on each grape]
4. transform preprocessed spectra into latent variables in a reduced dimensional latent space via, e.g., the previously trained autoencoder
5. predict perceptual characteristics of finished wine from the latent variables via the previously trained machine learning model
6. decide how to treat grape product based on prediction from operation 5

### Training—Example 2

Train an autoencoder or similar system using only optical spectra acquired from wine training samples. This training is unsupervised, semi-supervised, or self-supervised. It identifies relevant patterns in the wine spectral data and defines the latent space.

Train a neural network or similar system using (a) the transformed wine data from the latent space and (b) chemical and/or perception data about the wine. This is a supervised training process, with the information about the wine serving as labels.

### Inference Scenario 3 (Based on Example 2 Training)

1. obtain wine made from grapes that were possibly exposed to smoke
2. prepare sample [e.g., apply SERS material to wine]
3. make multiple measurements of wine sample to generate a set of spectra for the sample
4. transform preprocessed spectra into points in a reduced dimensional latent space via, e.g., an autoencoder
5. predict organic compound profile of finished wine from the transformed spectral data via a trained machine learning model
6. decide how to treat wine based on prediction from operation 5

### Inference Scenario 4 (Based on Example 2 Training)

1. obtain wine made from grapes that were possibly exposed to smoke
2. prepare sample [e.g., apply SERS material to wine]
3. make multiple measurements of wine sample to generate a set of spectra for the sample
4. transform preprocessed spectra into points in a reduced dimensional latent space via, e.g., an autoencoder
5. predict perceptual characteristics of finished wine from the transformed spectral data via a trained machine learning model
6. decide how to treat wine based on prediction from operation 5

### Other Applications of Machine Learning Models

In one aspect, machine learning models may be implemented to authenticate a finished wine. Some implementations may also apply machine learning models to authenticate a grape product. In these implementations, machine learning models can output (directly or indirectly) grape variety, terroir details, appellation, vineyard, harvest year, microbiological contribution (e.g., Brettanomyces-induced perceptual characteristics), additives, and other characteristics or properties of a finished wine or a grape product. The machine learning models can take various input as explained above. These implementations may be used to provide various “fingerprints” for a wine or a grape product. The information can be used by wine merchants and others involved in the buying, selling, and rating of wines.

In another aspect, machine learning models may be implemented to direct wine making process. At various stages of the wine making process, optionally starting as early as the grapes on vines, samples are collected and analyzed using machine learning models. The models recommend (directly or indirectly) various aspects of wine making, such as additional grape sources (e.g., characteristics of other grapes to be added to a first grape source), harvest time, crushing conditions, must handling, time between crushing and fermentation, temperature prior to, during or after fermentation or incubation, incubation period, yeast, inoculum size, additives, pH, and substrate concentration. The models may recommend based on cost, target taste profile, perceptual score, chemical profile, and any combination thereof. Wine making process parameters are described as input variables to machine learning models above. Some implementations may use these wine making process parameters as outputs. In other implementations, these wine making process parameters as input to machine learning models are adjusted to search for target output (e.g., target taste profile, target chemical profile, target perceptual characteristics). Other implementations may use machine learning models as a component in a process that recommends these wine making process parameters.

In a further aspect, machine learning models may be implemented to direct flavor engineering—a process to modify a grape product, a wine making process, or a wine to achieve a wine having a desired flavor profile. In some implementations, flavor engineering modifies a wine by various methods, e.g., blending, reverse osmosis, adding additives, processing, etc. The machine learning model analyzes a grape product and/or the final wine and provides a recommendation (directly or indirectly) of a type and/or quantity of flavor engineering. Flavor engineering parameters may be used as input to machine learning models. Some implementations may use these flavor engineering parameters as output. In other implementations, these flavor engineering parameters as input to machine learning models are adjusted to search for target output (e.g., target taste profile, target chemical profile, target perceptual characteristics). Other implementations may use machine learning models as a component in a process that recommends these flavor engineering parameters.

### Computer Systems

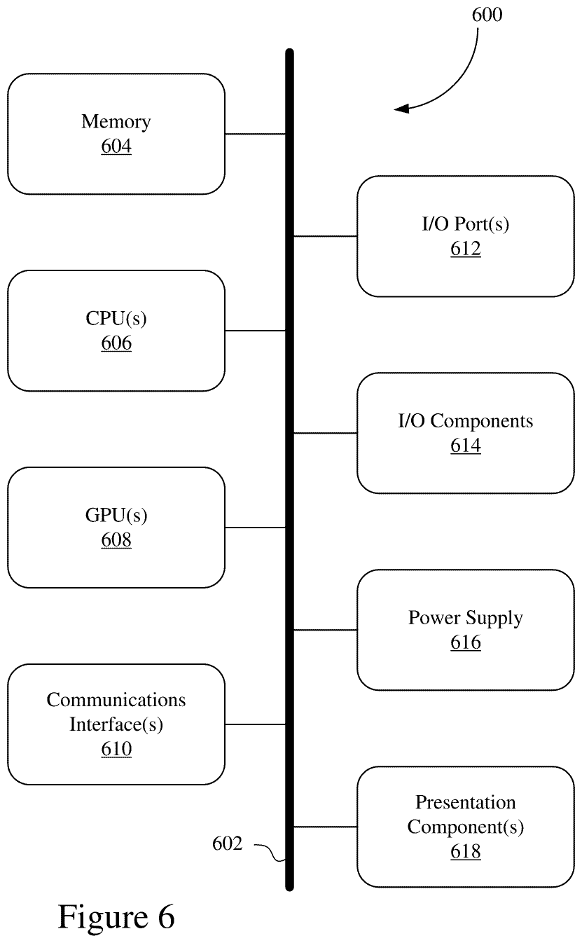

FIG. 6 is a block diagram of an example of the computing device or system 600 suitable for use in implementing computational aspects of some embodiments of the present disclosure. For example, device 600 may be suitable for implementing some or all operations associated with predicting smoke taint or other perceptual characteristic of a wine. For example, a computational system such as system 600 may be employed to receive optical signals and/or other input about a grape product or other sample, extract features from such input, and predict smoke taint or other perceptual characteristic of a wine as disclosed herein. In other examples, a computational system such as system 600 may be employed to receive training data about grape products (e.g., optical signals from the products, perceptual characteristics such as smoke taint or Brettanomyces contribution in the finished wine, and/or chemical composition information about the grape products), train a feature extractor, and/or train machine learning model that employs latent variables and predicts smoke taint, Brettanomyces contribution, or other perceptual characteristic of a wine.

Computing device 600 may include a bus 602 that directly or indirectly couples the following devices: memory 604, one or more central processing units (CPUs) 606, one or more graphics processing units (GPUs) 608, a communication interface 610, input/output (I/O) ports 612, input/output components 614, a power supply 616, and one or more presentation components 618 (e.g., display(s)). In addition to CPU 606 and GPU 608, computing device 600 may include additional logic devices that are not shown in FIG. 6, such as but not limited to an image signal processor (ISP), a digital signal processor (DSP), a deep learning processor (DLP), an ASIC, an FPGA, or the like.

Although the various blocks of FIG. 6 are shown as connected via the bus 602 with lines, this is not intended to be limiting and is for clarity only. For example, in some embodiments, a presentation component 618, such as a display device, may be considered an I/O component 614 (e.g., if the display is a touch screen). As another example, CPUs 606 and/or GPUs 608 may include memory (e.g., the memory 604 may be representative of a storage device in addition to the memory of the GPUs 608, the CPUs 606, and/or other components). In other words, the computing device of FIG. 6 is merely illustrative. Distinction is not made between such categories as “workstation,” “server,” “laptop,” “desktop,” “tablet,” “client device,” “mobile device,” “hand-held device,” “electronic control unit (ECU),” “virtual reality system,” and/or other device or system types, as all are contemplated within the scope of the computing device of FIG. 6.

Bus 602 may represent one or more busses, such as an address bus, a data bus, a control bus, or a combination thereof. The bus 602 may include one or more bus types, such as an industry standard architecture (ISA) bus, an extended industry standard architecture (EISA) bus, a video electronics standards association (VESA) bus, a peripheral component interconnect (PCI) bus, a peripheral component interconnect express (PCIe) bus, and/or another type of bus.

Memory 604 may include any of a variety of computer-readable media. The computer-readable media may be any available media that can be accessed by the computing device 600. The computer-readable media may include both volatile and nonvolatile media, and removable and non-removable media. By way of example, and not limitation, the computer-readable media may comprise computer-storage media and/or communication media.

The computer-storage media may include both volatile and nonvolatile media and/or removable and non-removable media implemented in any method or technology for storage of information such as computer-readable instructions, data structures, program modules, and/or other data types. For example, memory 604 may store computer-readable instructions (e.g., that represent a program(s) and/or a program element(s), such as an operating system. Computer-storage media may include, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium which can be used to store the desired information and which can be accessed by computing device 600. As used herein, computer storage media does not comprise signals per se.

The communication media may embody computer-readable instructions, data structures, program modules, and/or other data types in a modulated data signal such as a carrier wave or other transport mechanism and includes any information delivery media. The term “modulated data signal” may refer to a signal that has one or more of its characteristics set or changed in such a manner as to encode information in the signal. By way of example, and not limitation, the communication media may include wired media such as a wired network or direct-wired connection, and wireless media such as acoustic, RF, infrared and other wireless media. Combinations of any of the above should also be included within the scope of computer-readable media.

CPU(s) 606 may be configured to execute the computer-readable instructions to control one or more components of the computing device 600 to perform one or more of the methods and/or processes described herein. CPU(s) 606 may each include one or more cores (e.g., one, two, four, eight, twenty-eight, seventy-two, etc.) that are capable of handling a multitude of software threads simultaneously. CPU(s) 606 may include any type of processor and may include different types of processors depending on the type of computing device 600 implemented (e.g., processors with fewer cores for mobile devices and processors with more cores for servers). For example, depending on the type of computing device 600, the processor may be an ARM processor implemented using Reduced Instruction Set Computing (RISC) or an x86 processor implemented using Complex Instruction Set Computing (CISC). Computing device 600 may include one or more CPUs 606 in addition to one or more microprocessors or supplementary co-processors, such as math co-processors.

GPU(s) 608 may be used by computing device 600 to render graphics (e.g., 3D graphics). GPU(s) 608 may include many (e.g., tens, hundreds, or thousands) of cores that are capable of handling many software threads simultaneously. GPU(s) 608 may generate pixel data for output images in response to rendering commands (e.g., rendering commands from CPU(s) 606 received via a host interface). GPU(s) 608 may include graphics memory, such as display memory, for storing pixel data. The display memory may be included as part of memory 604. GPU(s) 608 may include two or more GPUs operating in parallel (e.g., via a link). When combined, each GPU 608 can generate pixel data for different portions of an output image or for different output images (e.g., a first GPU for a first image and a second GPU for a second image). Each GPU can include its own memory or can share memory with other GPUs.

In examples where the computing device 600 does not include the GPU(s) 608, the CPU(s) 606 may be used to render graphics.

Communication interface 610 may include one or more receivers, transmitters, and/or transceivers that enable computing device 600 to communicate with other computing devices via an electronic communication network, included wired and/or wireless communications. Communication interface 610 may include components and functionality to enable communication over any of a number of different networks, such as wireless networks (e.g., Wi-Fi, Z-Wave, Bluetooth, Bluetooth LE, ZigBee, etc.), wired networks (e.g., communicating over Ethernet), low-power wide-area networks (e.g., LoRaWAN, SigFox, etc.), and/or the internet.

I/O ports 612 may enable the computing device 600 to be logically coupled to other devices including I/O components 614, presentation component(s) 618, and/or other components, some of which may be built in to (e.g., integrated in) computing device 600. Illustrative I/O components 614 include a microphone, mouse, keyboard, joystick, track pad, satellite dish, scanner, printer, wireless device, etc. I/O components 614 may provide a natural user interface (NUI) that processes air gestures, voice, or other physiological inputs generated by a user. In some instances, inputs may be transmitted to an appropriate network element for further processing. An NUI may implement any combination of speech recognition, stylus recognition, facial recognition, biometric recognition, gesture recognition both on screen and adjacent to the screen, air gestures, head and eye tracking, and touch recognition (as described in more detail below) associated with a display of computing device 600. Computing device 600 may be include depth cameras, such as stereoscopic camera systems, infrared camera systems, RGB camera systems, touchscreen technology, and combinations of these, for gesture detection and recognition. Additionally, computing device 600 may include accelerometers or gyroscopes (e.g., as part of an inertia measurement unit (IMU)) that enable detection of motion. In some examples, the output of the accelerometers or gyroscopes may be used by computing device 600 to render immersive augmented reality or virtual reality.

Power supply 616 may include a hard-wired power supply, a battery power supply, or a combination thereof. Power supply 616 may provide power to computing device 600 to enable the components of computing device 600 to operate.

Presentation component(s) 618 may include a display (e.g., a monitor, a touch screen, a television screen, a heads-up-display (HUD), other display types, or a combination thereof), speakers, and/or other presentation components. Presentation component(s) 618 may receive data from other components (e.g., GPU(s) 608, CPU(s) 606, etc.), and output the data (e.g., as an image, video, sound, etc.).

The disclosure may be described in the general context of computer code or machine-usable instructions, including computer-executable instructions such as program modules, being executed by a computer or other machine, such as a cell phone, tablet, or other handheld device. Generally, program modules including routines, programs, objects, components, data structures, etc., refer to code configured to perform particular tasks or implement particular data types. The disclosure may be practiced in a variety of system configurations, including hand-held devices, consumer electronics, general-purpose computers, more specialty computing devices, etc. The disclosure may also be practiced in distributed computing environments where tasks are performed by remote-processing devices that are linked through a communications network. Some disclosed embodiments may be implemented, at least in part, using cloud-based resources.

### Experimental

Using Au NP paper substrates from Metrohm, SERS enhancements were found for guaiacols using an experimental setup of 785 nm system as described below. Using sample concentrations of 1000, 500, and 100 ppm, the intensity easily passed a discrimination test. Particularly, signal at 579 cm⁻¹ went from 200 counts using classical Raman of solutions of 81000 ppm, to 8000 counts of 100 ppm guaiacol solution. In addition, a large enhancement was observed in 1160 and 1265 cm⁻¹. A sub 1 ppm guaiacol detection limit is achievable based on these peaks.

Separately, grape juice and wine samples were tested using the same type of substrate. Using H2O and synthetic juice over NP substrates as baselines, we found three new peaks not coming from fructose, glucose, sucrose, malic acid, or tartaric acid. One of the peaks, at 730 cm⁻¹, is extremely strong and sharp. The other peaks are at 960 cm⁻¹ and 1315 cm⁻¹. These peaks are consistent with the result reported by Zanuttin [1]. Their paper assigned these peaks as coming from adenine. Qu also reported these peaks from wine interacting with SERS and confirmed its origin using adenine, catechin, and condensed tannin [2]. However, this is the first time that adenine, or catechin was reported from SERS spectra of grape juice. We have achieved system stability on par with the most recent publications of wine and SERS. These peaks are not related to related to phenols.

With Au NP, 785 nm CW Raman is no longer limited by fluorescence effect. Also, irrelevant peaks such as those from ethanol are no longer interfering. Collectively, these advantages of SERS-Raman analysis create suitable conditions for feature extraction and machine-learning (ML).

### Guaiacol Detection Using Raman SERS

### Materials Used

o-Guaiacol: 51200 ppm (pure guaiacol), 81000 ppm, 1000 ppm, 500 ppm, 100 ppm.

Other than pure o-guaiacol, the other compositions were guaiacol (Sigma-Aldrich) diluted in synthetic wine made by dissolving 3.5 g of L-tartaric acid (Alfa Aesar) in 1 L of 12% ethanol solution and adjusting the pH to 3.5 with 1 M NaOH.

Deionized H₂O

Metrohm SERS substrates, each paper strip was cut into 9 pieces

Red 3D-printed sample holder for each substrate piece

Regarding the Metrohm SERS substrates, these were prepared by printing gold nanoparticles (AuNPs) on chromatography paper via ink-jet printers. The gold colloids were prepared via Lee-Meisel method and subsequently concentrated. Despite microscopic variability, excellent reproducibility was achieved with probing Raman beam.

### Experimental Details

5 μL of the above-described guaiacol solutions were used. The duration from dispense to measurement was controlled to ˜0.5 mins.

A Metrohm Snowy Range Sierra Raman spectrometer was equipped with a 785 nm laser operating in continuous wave mode. Its thermoelectrically cooled 2048-pixel detector has a spectral range of 200-2000 cm⁻¹ with spectral resolution is 4 cm⁻¹. Right after 5 μL of sample solution was dropped onto the SERS substrate and allowed to saturate for 30 seconds, spectral acquisition started at the focal position. The laser was set at 10 mW power, 10 s integration time, and a multi-scan option of 5 with orbital rastering on.

### Results

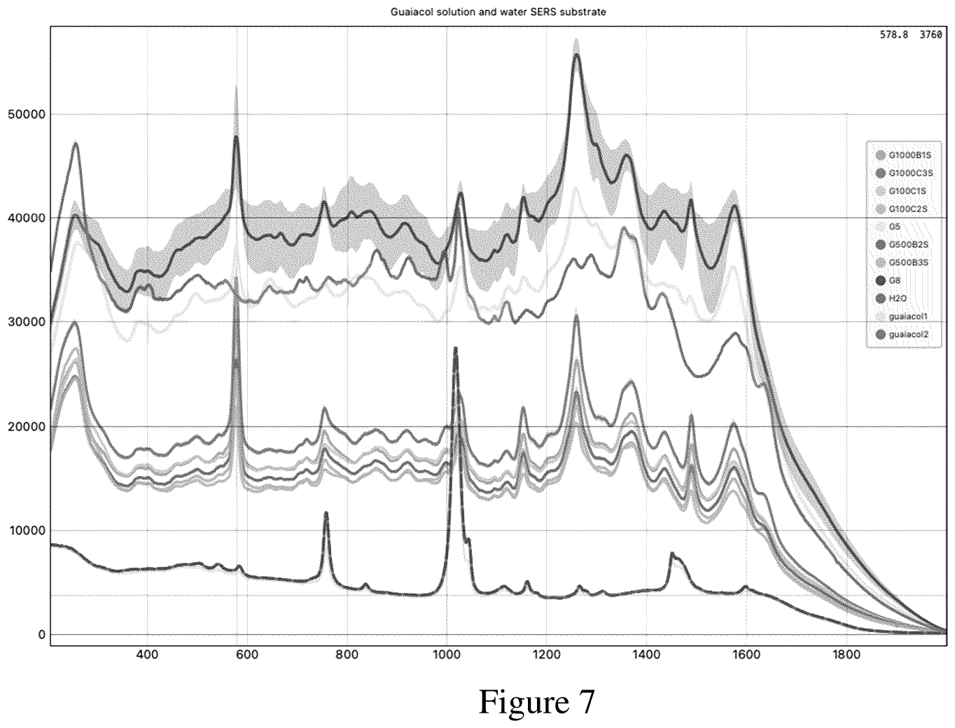

As depicted in FIG. 7, at the bottom of the graph, one can see guaiacol 1 and 2. They are the high concentration solutions, and the signal was captured without SERS. When these solutions are applied to the SERS substrate, they are labeled G8 and G5. There are many smaller peaks, which can be traced to H₂O on SERS (G1000B1S curve). Guaiacol samples of concentrations at 1000, 500, and 100 ppm were measured in pairs of samples (G1000xxS, G500xxS, G100xxS). At these low concentrations, enhancement at 578, 1159 and 1261 cm⁻¹ were clearly visible and likely are related to the bending and breathing mode of the aromatic compounds. There is a background contribution that obscured the relationship of these measurements. The shaded regions emanating from the plots in FIG. 7 are from multiple measurements, and the range depicts the variation while the average is at the center.

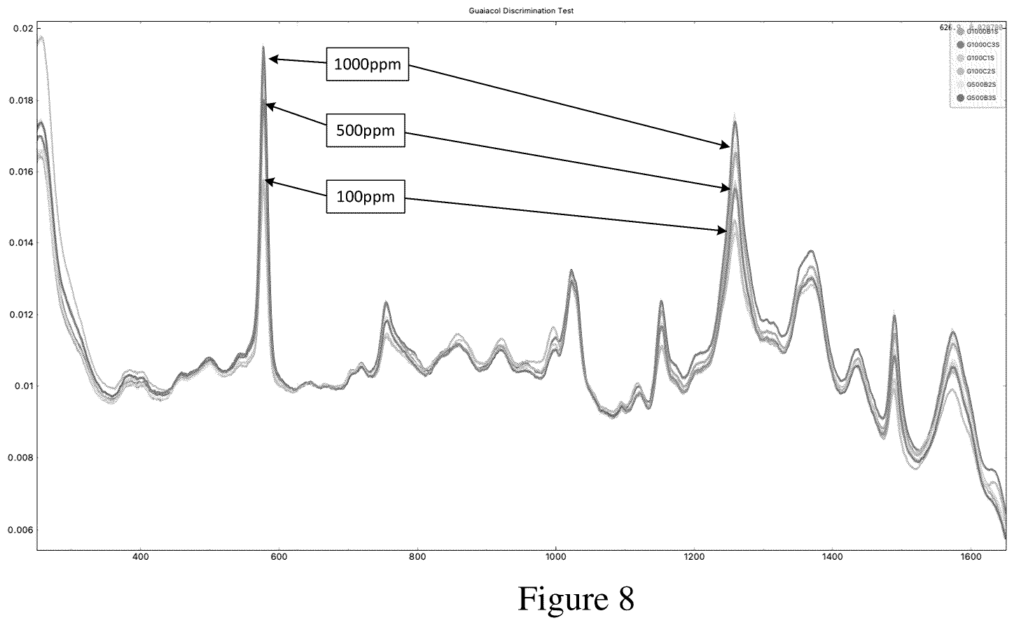

In FIG. 8, this background has been subtracted out. After background subtraction, the grouping of the three regions with two measurements each becomes clear. They are the result of guaiacol concentration. Due to the size of the peaks, the lower guaiacol concentration limit has not been reached. A detection limit of about 10, about 1, or even about 0.1 ppm may be achieved.

### Grape Juice and Wine Interaction With SERS Substrate

### Materials Used

Juice samples were prepared from 2022 grape season.

Wine samples: These were produced by Oregon wineries with no smoke exposure, no barrel aging, or other forms of contamination. The wines were gathered from across Oregon between 2013-2015 and stored in a cold room at Oregon State University under conditions to prevent oxidation.

Deionized H₂O

Synthetic juice (with Fructose, glucose, malic acid, tartaric acid, and citric acid)

Metrohm SERS substrates, each cut into 9 pieces

Red 3D-printed sample holder

### Experimental Details

Use 5 μL of solution from above samples or juice

Control the duration from dispense to measurement˜0.5 mins.

Intensity setting: 15, Raster On, integration time 5-20 sec, Multi-acquisition 20.

Micrometer at 0, sample holder place on top of 96-well plate

Visual alignment of X/Y

### Results

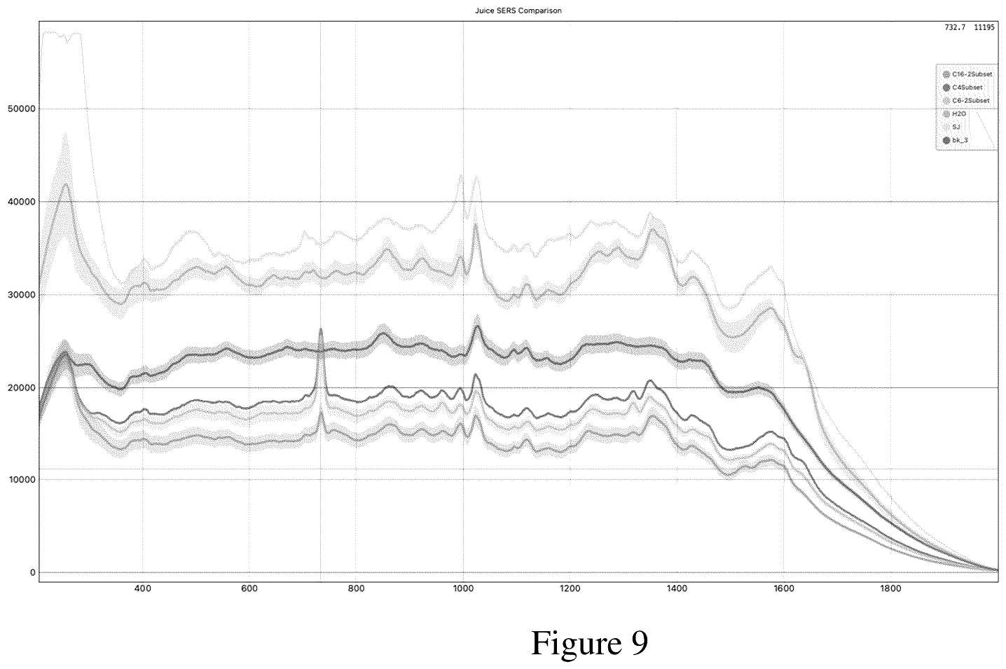

As depicted in FIG. 9, starting from blanket SERS substrate (bk_3), we added H₂O and synthetic (SJ) juice to find there was little to no identifiable features from sugars and acids. When the actual grape juice was presented, the peaks of 733, 961, 1324 cm⁻¹ starts to show up. This is consistent with the findings from Zanuttin [1] and Qu [2] regarding wines. They are of adenine or catechin origin. [3]

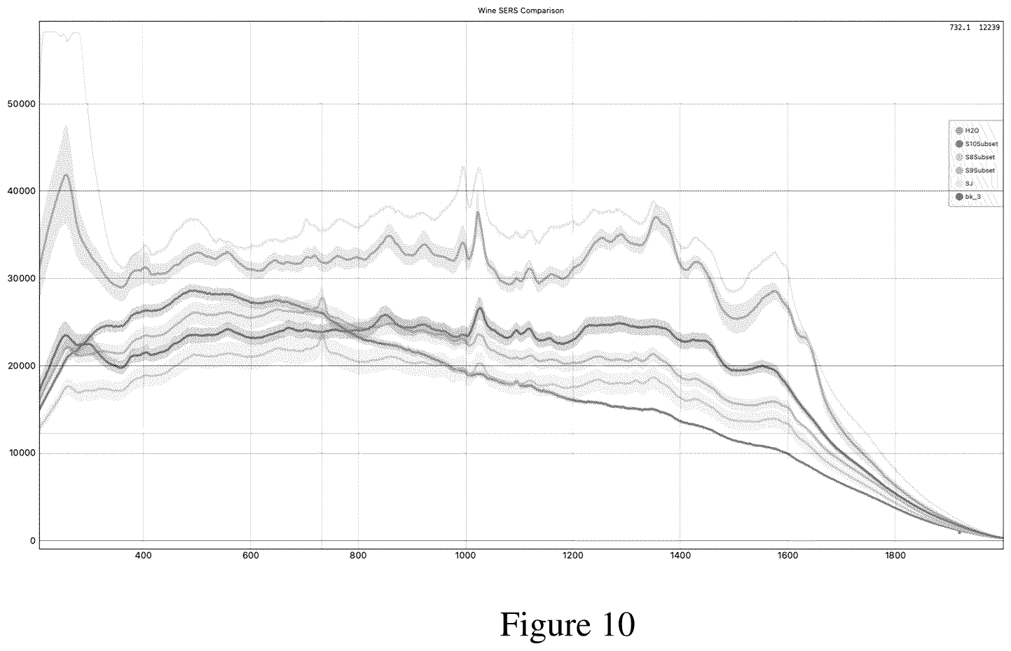

As depicted in FIG. 10, other than sample S10, wine sample S8 and S9 showed consistent results as the grape samples. A lack of fluorescence and catechin or adenine peaks. They are only weaker in intensity relative to the juice samples. Sample S10 is different most likely due to the chemical extraction and anthocyanin, which introduces a fluorescent background. One may use these spectra to conduct ML and correlate to reported guaiacol values. Natural variations of smoke response may be reflected in the relationship of all these peaks, as well as the magnitude and width of the other Raman peaks.

### High Sensitivity Detection of Guaiacol With SERS Substrate

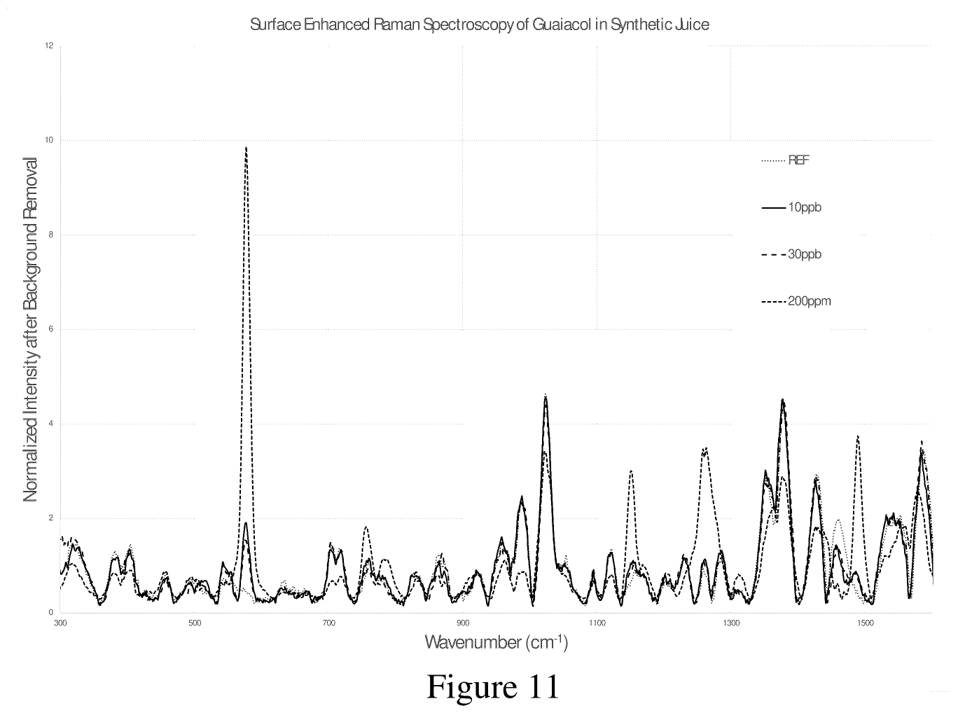

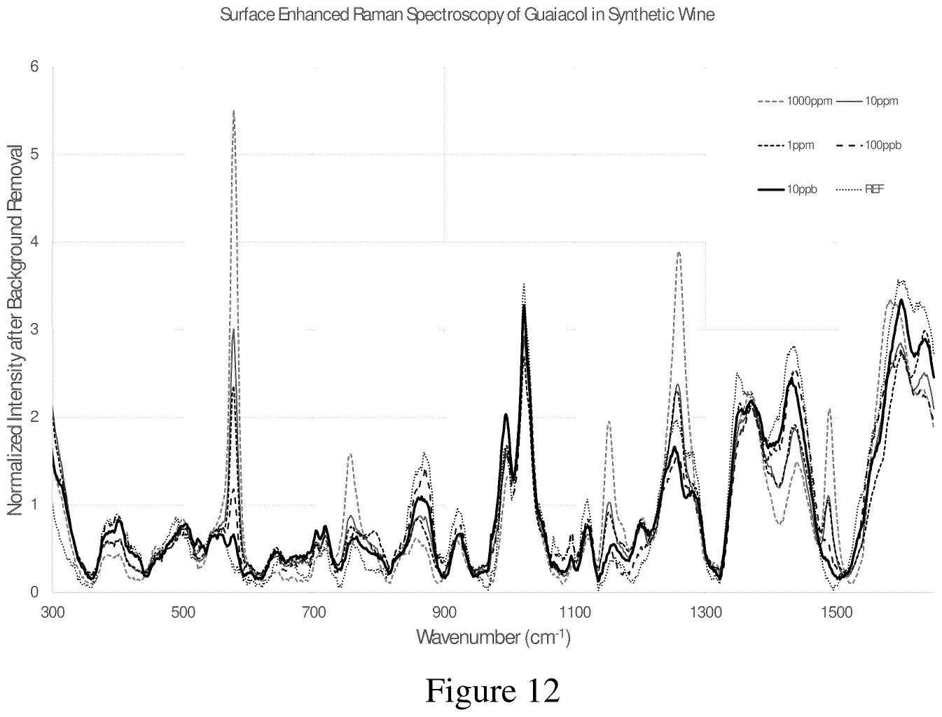

The data presented in FIGS. 11 and 12 was generated using an experimental set up as described above for FIGS. 7-10, including the Raman SERS system. FIG. 11 illustrates results for synthetic juice with different concentrations of guaiacol, and FIG. 12 illustrates results for synthetic wine with guaiacol. The synthetic juice was made with H₂O, fructose, glucose, tartaric acid, malic acid in mass ratio of 82:10:5:5:3. The synthetic wine was made by dissolving 3.5 g of L-tartaric acid (Alfa Aesar) in 1 L of 12% ethanol solution and adjusting the pH to 3.5 with 1 M NaOH.

Note from the graphs in FIGS. 11 and 12 that the system identified SERS-enhanced guaiacol peaks at 577, 756, 1151, 1259, 1369, 1489, and 1571 cm⁻¹. These peaks correspond to the following vibrations:

| Wavenumber | |
|---|---|
| (cm⁻¹) | Vibration Assignment |
| 577: | Aromatic ring deformation or bending |
| 756: | Aromatic C—H out-of-plane bending |
| 1151: | C—H in-plane bending/C—O stretching |
| 1259: | C—O stretching (methoxy/phenolic group) |
| 1369: | O—H bending (phenol)/Aromatic C—H deformation |
| 1489: | Aromatic C═C stretching |
| 1571: | Aromatic C═C stretching (higher energy mode) |

References, each of which is incorporated herein by reference in its entirety:
[1] Zanuttin, F.; Gurian, E.; Ignat, I.; Fornasaro, S.; Calabretti, A.; Bigot, G.; Bonifacio, A. “Characterization of white wines from north-eastern Italy with surface-enhanced Raman spectroscopy.” Talanta 2019, 203, 99-105.
[2] Qu, Y. Q.; Tian, Y.; Chen, Y. H.; He, L. L. “Chemical profiling of red wines using surface-enhanced Raman spectroscopy (SERS).” Anal. Methods 2020, 12, 1324-1332.
[3] Pompeu, D. R.; Larondelle, Y.; Rogez, H.; Abbas, O.; Pierna, J. A. F.; Baeten, V. Characterization and discrimination of phenolic compounds using fourier transform Raman spectroscopy and chemometric tools. Biotechnol. Agron. Soc. Environ. 2018, 22, 13-28.

### Conclusion

Although the foregoing embodiments have been described in some detail for purposes of clarity of understanding, it will be apparent that certain changes and modifications may be practiced within the scope of the appended claims. It should be noted that there are many alternative ways of implementing the processes, systems, and apparatus of the present embodiments. Accordingly, the present embodiments are to be considered as illustrative and not restrictive, and the embodiments are not to be limited to the details given herein.

What is claimed is:

1. A method of predicting smoke taint, the method comprising:
receiving an optical signal from a sample of a grape product or a wine, wherein the optical signal comprises a spectrum having characteristics influenced by chemical components of the grape product or the wine;
extracting features from the optical signal by transforming information in the optical signal to a latent space, which latent space has reduced dimensions compared to dimensions of the optical signal; and
providing the features to a machine learning model that provides (i) chemical composition information in the grape product or the wine, wherein the chemical composition information includes information about one or more compounds that are associated smoke taint, and/or (ii) one or more perceptual characteristics of a finished wine produced from the grape product or of the wine, wherein the perceptual characteristics indicate whether the finished wine produced from the grape product or the wine exhibits a smoke taint perceptual characteristic.

2. The method of claim 1, wherein the sample comprises one or more grapes used to produce the finished wine.

3. The method of claim 1, wherein the sample comprises must or juice used to produce the finished wine.

4. The method of claim 1, wherein the sample comprises the wine.

5. The method of claim 1, further comprising obtaining the optical signal from the sample comprising the grape product or the wine.

6. The method of claim 1, wherein the optical signal is a signal from Raman spectroscopy performed on the grape product or the wine.

7. The method of claim 6, wherein Raman spectroscopy is a Surface Enhanced Raman Spectroscopy (SERS).

8. The method of claim 7, further comprising contacting the sample with a nanoscale material that enhances Raman signals from the sample.

9. The method of claim 8, further comprising: contacting the sample with multiple nanostructures, and obtaining a Raman signal for each nanostructure and sample combination.

10. The method of claim 1, wherein the optical signal comprises information from at least 10 wavelengths.

11. The method of claim 1, further comprising providing information from a portion of a grape plant that is not a grape to the machine learning model, wherein the grape plant produced grapes for the grape product or the wine.

12. The method of claim 11, wherein the portion of the grape plant comprises a leaf of the grape plant.

13. The method of claim 1, further comprising providing information from an electronic sensor of volatile organic compounds to the machine learning model.

14. The method of claim 1, further comprising providing information from infrared, fluorescence, and/or visible spectra of the grape product or the wine to the machine learning model.

15. The method of claim 1, further comprising providing information about a wine making process for producing the wine or the finished wine to the machine learning model.

16. The method of claim 15, wherein the information about the wine making process is selected from a group consisting of: grape sources, harvest time, crushing conditions, must handling, time between crushing and fermentation, temperature prior to, during or after fermentation or incubation, incubation period, yeast, inoculum size, additives, pH, substrate concentration, and any combinations thereof.

17. The method of claim 1, wherein extracting features comprises providing the optical signal to a variational autoencoder or a transformer model trained using training data comprising training optical signals from smoke tainted grape products or wines.

18. The method of claim 1, wherein the machine learning model is trained using training data comprising training mass spectrometry data obtained from smoke tainted grape products or wines.

19. The method of claim 18, wherein the training mass spectrometry data are obtained using a technique selected from a group consisting of gas chromatography-mass spectroscopy (GC-MS), gas chromatography-tandem mass spectroscopy (GC-MS-MS), high performance liquid chromatography-mass spectroscopy (HPLC-MS), high performance liquid chromatography-tandem mass spectroscopy (HPLC-MS-MS), high performance liquid chromatography-diode array detector-mass spectroscopy (HPLC-DAD-MS), and any combinations thereof.

20. The method of claim 18 or 19, wherein the machine learning model provides chemical composition information in the grape product or the wine corresponding to the training mass spectrometry data.

21. The method of claim 1, further comprising:
producing multiple optical signals from the sample;
extracting features from each of the multiple optical signals; and
combining the features from each of the multiple optical signal to produce one or more combined features of the multiple optical samples.

22. The method of claim 21, providing the features to the machine learning model comprises providing the combined features of the multiple optical samples to the machine learning model.

23. The method of claim 1, wherein the machine learning model is a neural network.

24. The method of claim 1, further comprising preprocessing the optical signal to normalize and/or reduce noise in the optical signal prior to extracting features from the optical signal.

25. The method of claim 1, wherein the chemical composition information includes information about a catechin, a tannin, an anthocyanin, a quercetin, a guaiacol, a cresol, a syringol, a glycoside of any of the foregoing, and any combination of the foregoing.

26. The method of claim 1, wherein the one or more compounds that are associated with smoke taint comprise a guaiacol, a cresol, a syringol, a glycoside of any of the foregoing, and any combination of the foregoing.

27. A method of training a machine learning model configured to predict smoke taint in a wine, the method comprising:
receiving training data for each of a plurality of samples, each sample comprising a grape product and/or a wine, wherein the training data for each sample comprises (a) an optical signal generated from the sample, and (b) (i) chemical composition information in the grape product, a finished wine produced from the grape product, or the wine, wherein the chemical composition information includes information about one or more compounds that are associated with smoke taint, and/or (ii) one or more perceptual characteristics of the finished wine produced from the grape product or of the wine, wherein the perceptual characteristics indicate whether the finished wine produced from the grape product or the wine exhibits a smoke taint perceptual characteristic;
training a feature extractor using at least a portion of the training data, wherein the feature extractor is trained to extract features from the optical signals generated from the training samples by transforming information in the optical signals to a latent space, which latent space has reduced dimensions compared to dimensions of the optical signals; and
training a machine learning model, using at least a portion of the training data and at least features extracted from the training data by the feature extractor, wherein the machine learning model is trained to predict (i) the chemical composition information in the grape product, the finished wine produced from the grape product, or the wine, and/or (ii) one or more perceptual characteristics of the finished wine produced from the grape product or of the wine.

28. The method of claim 27, wherein the plurality of samples comprises a plurality of grapes.

29. The method of claim 27, wherein the plurality of samples comprises must or juice.

30. The method of claim 27, wherein the plurality of samples comprises a grape product spiked with one or more smoke taint compounds and/or a wine spiked with one or more smoke taint compounds.

31. The method of claim 27, wherein the optical signals from the samples comprise Raman spectra.

32. The method of claim 31, wherein the Raman spectra were obtained using a Surface Enhanced Raman Spectroscopy (SERS).

33. The method of claim 27, wherein the optical signals from the samples each comprise information from at least 10 wavelengths.

34. The method of claim 27, wherein the training data for each of the plurality of samples further comprises information from a portion of a grape plant that is not a grape, wherein the grape plant produced grapes for the grape product or the wine.

35. The method of claim 34, wherein the portion of the grape plant comprises a leaf of the grape plant.

36. The method of claim 27, wherein the training data further comprises information from an electronic sensor of volatile organic compounds.

37. The method of claim 27, wherein the training data further comprises information from fluorescence spectra, infrared spectra and/or visible spectra of the grape produce or the wine.

38. The method of claim 27, wherein training the feature extractor comprises training a variational autoencoder or a transformer model using the optical signals generated from the samples.

39. The method of claim 27, wherein the machine learning model is a neural network.

40. The method of claim 27, wherein the chemical composition information comprises information about a catechin, a tannin, an anthocyanin, a quercetin, a guaiacol, a cresol, a syringol, a glycoside of any of the foregoing, and any combination of the foregoing.

41. The method of claim 27, wherein the one or more compounds that cause smoke taint comprise a guaiacol, a-cresol, a syringol, a glycoside of any of the foregoing, and any combination of the foregoing.

42. A method of predicting perceptual characteristics, the method comprising:
receiving an optical signal from a sample of a grape product or a wine, wherein the optical signal comprises a spectrum having characteristics influenced by chemical components of the grape product or the wine;
extracting features from the optical signal by transforming information in the optical signal to a latent space, which latent space has reduced dimensions compared to dimensions of the optical signal; and
providing the features to a machine learning model that outputs (i) chemical composition information in the grape product or the wine, wherein the chemical composition information includes information about one or more compounds that are associated with one or more perceptual characteristics, and/or (ii) the one or more perceptual characteristics of a finished wine produced from the grape product or of the wine.

43. The method of claim 42, wherein the one or more perceptual characteristics comprise a perceptual characteristic selected from the group consisting of a taste, an aroma, a mouthfeel, an appearance, and any combinations thereof.

44. The method of claim 42, wherein the one or more perceptual characteristics comprise a smoke taint perceptual characteristic.

45. The method of claim 42, wherein the one or more perceptual characteristics comprise a perceptual characteristic associated with one or more chemical byproducts of an organism, and wherein the chemical composition information comprises information about the one or more chemical byproducts of the organism.

46. The method of claim 45, wherein the organism comprises Brettanomyces, and wherein the chemical composition information comprises information about 4-ethylphenol, 4-ethylguaiacol, 4-ethylcatechol, and/or 4-propylguaiacol.

47. The method of claim 45 or 46, further comprising repeating the method for multiple samples obtained at multiple stages in a wine making process.

48. The method of claim 47, further comprising using the output of the machine learning model at the multiple stages of the wine making process to account for potential variations in the presence or concentration of Brettanomyces during the wine making process.

49. The method of claim 42, wherein the one or more perceptual characteristics comprise a perceptual characteristic indicating whether or not consumers favorably perceives the finished wine produced from the grape product or the wine.

50. The method of claim 42, wherein the one or more perceptual characteristics comprise a favorability score indicating how favorably consumers consider the finished wine produced from the grape product or the wine.

51. The method of claim 42, wherein the one or more perceptual characteristics comprise one or more metrics of the finished wine or a score representing the one or more metrics of the finished wine.

52. The method of claim 42, wherein the machine learning model is configured to output information selected from a group consisting of: grape variety, terroir details, appellation, vineyard, harvest year, additives, and other characteristics or properties of a finished wine or a grape product, and any combinations thereof.

53. The method of claim 42, wherein the machine learning model is configured to output a recommendation of one or more wine making process parameters.

54. The method of claim 53, wherein the one or more wine making process parameters is selected from a group consisting of: grape sources, harvest time, crushing conditions, must handling, time between crushing and fermentation, temperature prior to, during or after fermentation or incubation, incubation period, yeast, inoculum size, additives, pH, substrate concentration, and any combinations thereof.

55. The method of claim 42, the machine learning model is configured to output a recommendation of a type and/or a parameter of flavor engineering process.

56. The method of claim 42, wherein the chemical composition information comprises information about a catechin, tannin, anthocyanin, terpinol, linalool, geraniol, α-terpineol, citronelol, nerol, nor-isoprenoid, β-damsascenone, β-ionone, α-ionone, ethyl cinnamate, ethyl dihydrocinnamate, hexanol, Z-3-hexenol, E-2-hexenol, ethanol, fusel alcohol, isobutanol, 2 and 3-methylbutanol, isoamylalcohol, β-phenylethanol, methionol, fusel alcohol acetate, isobutyl acetate, isoamyl acetate, hexyl acetate, phenylethyl acetate, fatty acid, acetic acid, butyric acid, hexanoic acid, octanoic acid, decanoic acid, ethyl acetate, ethyl butyrate, ethyl hexanoate, ethyl octanoate, ethyl decanoate, isobutyric acid, 2-methylbutyric acid, 3-methylbutyric acid, isovaleric acid, ethyl isobutyrate, ethyl 2-methylbutyrate, ethyl 3-methylbutyrate, ethyl isovalerate, carbonyl, lactone, diacetyl, 2,3-pentanedione, acetoine, γ-butyrolactone, ethyl lactate, diethyl succinate, Z-whiskylactone, E-whiskylactone, o and m-cresol, guaiacol, 4-methylguaiacol, eugenol, E-isoeugenol, 2,6-dimethoxyphenol, 4-allyl-2,6-dimethoxyphenol, vanillin, acetovanillone, propiovanillone, ethylvanillate, methylvanillate, furfural, 5-methylfurfural, 4-ethylphenol, 4-ethylguaiacol, 4-propylguaiacol, γ-lactones, γ-octalactone, γ-nonalactone, γ-decalactone, γ-undecalactone, γ-dodecalactone, 4-vinylphenol, 4-vinylguaiacol, and any combination of the foregoing.

57. The method of claim 42, wherein the sample comprises one or more grapes used to produce the finished wine.

58. The method of claim 42, wherein the sample comprises must or juice used to produce the finished wine.

59. The method of claim 42, wherein the sample comprises the wine.

60. The method of claim 42, further comprising obtaining the optical signal from the sample comprising the grape product or the wine.

61. The method of claim 42, wherein the optical signal is a signal from Raman spectroscopy performed on the grape product or the wine.

62. The method of claim 61, wherein Raman spectroscopy is a Surface Enhanced Raman Spectroscopy (SERS).

63. The method of claim 62, further comprising contacting the sample with a nanoscale material that enhances Raman signals from the sample.

64. The method of claim 42, further comprising providing information from an electronic sensor of volatile organic compounds to the machine learning model.

65. The method of claim 42, further comprising providing information from infrared, fluorescence, and/or visible spectra of the grape produce or the wine to the machine learning model.

66. The method of claim 42, wherein extracting features comprises providing the optical signal to a variational autoencoder or a transformer model trained using training optical signals from training data.

67. The method of claim 42, wherein the machine learning model is a neural network.

68. The method of claim 42, wherein extracting features comprises providing the optical signal to a variational autoencoder or a transformer model trained using training data comprising training optical signals from training samples of grape products and/or wines.

69. The method of claim 42, wherein the machine learning model is trained using training data comprising training mass spectrometry data obtained from training samples of grape products and/or wines.

70. The method of claim 69, wherein the training mass spectrometry data are obtained using a technique selected from a group consisting of gas chromatography-mass spectroscopy (GC-MS), gas chromatography-tandem mass spectroscopy (GC-MS-MS), high performance liquid chromatography-mass spectroscopy (HPLC-MS), high performance liquid chromatography-tandem mass spectroscopy (HPLC-MS-MS), high performance liquid chromatography-diode array detector-mass spectroscopy (HPLC-DAD-MS), and any combinations thereof.

71. The method of claim 69 or 70, wherein the machine learning model provides chemical composition information in the grape product or the wine corresponding to the training mass spectrometry data.

72. The method of claim 42, wherein the machine learning model is trained using training data comprising microbiological information indicating the presence of a microorganism.

73. The method of claim 72, wherein microbiological information is PCR results indicating the presence of the microorganism.

74. A method of training a machine learning model configured to predict perceptual characteristics of a wine, the method comprising:
receiving training data for each of a plurality of samples, each sample comprising a grape product and/or a wine, wherein the training data for each sample comprises (a) an optical signal generated from the sample, and (b) (i) chemical composition information in the grape product, a finished wine produced from the grape product, or the wine, wherein the chemical composition information includes information about one or more compounds that are associated with one or more perceptual characteristics, and/or (ii) the one or more perceptual characteristics of the finished wine produced from the grape product or of the wine;
training a feature extractor using at least a portion of the training data, wherein the feature extractor is trained to extract features from the optical signals generated from the training samples by transforming information in the optical signals to a latent space, which latent space has reduced dimensions compared to dimensions of the optical signals; and
training a machine learning model, using at least a portion of the training data and at least features extracted from the training data by the feature extractor, wherein the machine learning model is trained to predict (i) the chemical composition information in the grape product, the finished wine produced from the grape product, or the wine, and/or (ii) the one or more perceptual characteristics of the finished wine produced from the grape product or of the wine.

75. The method of claim 74, wherein the one or more perceptual characteristics comprise a perceptual characteristic selected from the group consisting of a taste, an aroma, a mouthfeel, an appearance, and any combinations thereof.

76. The method of claim 74, wherein the one or more perceptual characteristics comprise a smoke taint perceptual characteristic.

77. The method of claim 74, wherein the one or more perceptual characteristics comprise a Brettanomyces taint perceptual characteristic.

78. The method of claim 74, wherein the one or more perceptual characteristics comprise a perceptual characteristic indicating whether or not the finished wine produced from the grape product or the wine is favorably perceived by consumers.

79. The method of claim 74, wherein the one or more perceptual characteristics comprise a favorability score indicating how favorably the finished wine produced from the grape product or the wine is perceived by consumers.

80. The method of claim 74, wherein the chemical composition information comprises information about a catechin, tannin, anthocyanin, terpinol, linalool, geraniol, α-terpineol, citronelol, nerol, nor-isoprenoid, β-damsascenone, β-ionone, α-ionone, ethyl cinnamate, ethyl dihydrocinnamate, hexanol, Z-3-hexenol, E-2-hexenol, ethanol, fusel alcohol, isobutanol, 2 and 3-methylbutanol, isoamylalcohol, β-phenylethanol, methionol, fusel alcohol acetate, isobutyl acetate, isoamyl acetate, hexyl acetate, phenylethyl acetate, fatty acid, acetic acid, butyric acid, hexanoic acid, octanoic acid, decanoic acid, ethyl acetate, ethyl butyrate, ethyl hexanoate, ethyl octanoate, ethyl decanoate, isobutyric acid, 2-methylbutyric acid, 3-methylbutyric acid, isovaleric acid, ethyl isobutyrate, ethyl 2-methylbutyrate, ethyl 3-methylbutyrate, ethyl isovalerate, carbonyl, lactone, diacetyl, 2,3-pentanedione, acetoine, γ-butyrolactone, ethyl lactate, diethyl succinate, Z-whiskylactone, E-whiskylactone, o and m-cresol, guaiacol, 4-methylguaiacol, eugenol, E-isoeugenol, 2,6-dimethoxyphenol, 4-allyl-2,6-dimethoxyphenol, vanillin, acetovanillone, propiovanillone, ethylvanillate, methylvanillate, furfural, 5-methylfurfural, 4-ethylphenol, 4-ethylguaiacol, 4-propylguaiacol, γ-lactones, γ-octalactone, γ-nonalactone, γ-decalactone, γ-undecalactone, γ-dodecalactone, 4-vinylphenol, 4-vinylguaiacol, or any combination of the foregoing.

81. The method of claim 74, wherein the plurality of samples comprises a plurality of grapes.

82. The method of claim 74, wherein the plurality of samples comprises must or juice.

83. The method of claim 74, wherein the plurality of samples comprises a grape product spiked with one or more compounds causing the one or more perceptual characteristics and/or a wine spiked with one or more compounds causing the one or more perceptual characteristics.

84. The method of claim 74, wherein the plurality of samples comprises a grape product spiked with a microbial organism.

85. The method of claim 84, wherein the microbial organism is Brettanomyces.

86. The method of claim 74, wherein the optical signals from the samples comprise Raman spectra.

87. The method of claim 86, wherein the Raman spectra were obtained using a Surface Enhanced Raman Spectroscopy (SERS).

88. The method of claim 74, wherein the optical signals from the samples each comprise information from at least 10 wavelengths.

89. The method of claim 74, wherein the training data further comprises information from an electronic sensor of volatile organic compounds.

90. The method of claim 74, wherein the training data further comprises information from infrared spectra, fluorescence, and/or visible spectra of the grape produce or the wine.

91. The method of claim 74, wherein training the feature extractor comprises training a variational autoencoder or a transformer model using the optical signals generated from the samples.

92. The method of claim 74, wherein the machine learning model is a neural network.

93. A system for predicting perceptual characteristics of wine, the system comprising:
a processor and memory configured to:
receive an optical signal from a sample of a grape product or a wine, wherein the optical signal comprises a spectrum having characteristics influenced by chemical components of the grape product or the wine;
extract features from the optical signal by transforming information in the optical signal to a latent space, which latent space has reduced dimensions compared to dimensions of the optical signal; and
provide the features to a machine learning model that outputs (i) chemical composition information in the grape product or the wine, wherein the chemical composition information includes information about one or more compounds that are associated with one or more perceptual characteristics, and/or (ii) the one or more perceptual characteristics of a finished wine produced from the grape product or of the wine.

94. The system of claim 93, wherein the one or more perceptual characteristics comprise a perceptual characteristic selected from the group consisting of a taste, an aroma, a mouthfeel, an appearance, and any combinations thereof.

95. The system of claim 93, wherein the one or more perceptual characteristics comprise a smoke taint perceptual characteristic.

96. The system of claim 93, wherein the one or more perceptual characteristics comprise a perceptual characteristic associated with one or more chemical byproducts of an organism, and wherein the chemical composition information comprises information about the one or more chemical byproducts of the organism.

97. The system of claim 96, wherein the organism comprises Brettanomyces, and wherein the chemical composition information comprises information about 4-ethylphenol, 4-ethylguaiacol, 4-ethylcatechol, and/or 4-propylguaiacol.

98. The system of claim 93, wherein the one or more perceptual characteristics comprise one or more metrics of the finished wine or a score representing the one or more metrics of the finished wine.

99. The system of claim 93, wherein the machine learning model is configured to output a recommendation of one or more wine making process parameters.

100. The system of claim 93, further comprising a Raman spectrometer.

101. The system of claim 93, further comprising an electronic sensor of volatile organic compounds.

102. The system of claim 93, wherein the processor and memory are configured to extract the features by providing the optical signal to a variational autoencoder or a transformer model trained using training optical signals from training data.

103. The system of claim 93, wherein the machine learning model is a neural network.

104. The system of claim 93, wherein the processor and memory are configured to extract the features by providing the optical signal to a variational autoencoder or a transformer model trained using training data comprising training optical signals from training samples of grape products and/or wines.

105. The system of claim 93, wherein the machine learning model was trained using training data comprising training mass spectrometry data obtained from training samples of grape products and/or wines.

106. A system for training a machine learning model configured to predict perceptual characteristics of a wine, the system comprising:
a processor and memory configured to:
receive training data for each of a plurality of samples, each sample comprising a grape product and/or a wine, wherein the training data for each sample comprises (a) an optical signal generated from the sample, and (b) (i) chemical composition information in the grape product, a finished wine produced from the grape product, or the wine, wherein the chemical composition information includes information about one or more compounds that are associated with one or more perceptual characteristics, and/or (ii) the one or more perceptual characteristics of the finished wine produced from the grape product or of the wine;
train a feature extractor using at least a portion of the training data, wherein the feature extractor is trained to extract features from the optical signals generated from the training samples by transforming information in the optical signals to a latent space, which latent space has reduced dimensions compared to dimensions of the optical signals; and
train a machine learning model, using at least a portion of the training data and at least features extracted from the training data by the feature extractor, wherein the machine learning model is trained to predict (i) the chemical composition information in the grape product, the finished wine produced from the grape product, or the wine, and/or (ii) the one or more perceptual characteristics of the finished wine produced from the grape product or of the wine.

107. The system of claim 106, wherein the one or more perceptual characteristics comprise a perceptual characteristic selected from the group consisting of a taste, an aroma, a mouthfeel, an appearance, and any combinations thereof.

108. The system of claim 106, wherein the one or more perceptual characteristics comprise a smoke taint perceptual characteristic.

109. The system of claim 106, wherein the one or more perceptual characteristics comprise a Brettanomyces taint perceptual characteristic.

110. The system of claim 106, wherein the chemical composition information comprises information about a catechin, tannin, anthocyanin, terpinol, linalool, geraniol, α-terpineol, citronelol, nerol, nor-isoprenoid, β-damsascenone, β-ionone, α-ionone, ethyl cinnamate, ethyl dihydrocinnamate, hexanol, Z-3-hexenol, E-2-hexenol, ethanol, fusel alcohol, isobutanol, 2 and 3-methylbutanol, isoamylalcohol, β-phenylethanol, methionol, fusel alcohol acetate, isobutyl acetate, isoamyl acetate, hexyl acetate, phenylethyl acetate, fatty acid, acetic acid, butyric acid, hexanoic acid, octanoic acid, decanoic acid, ethyl acetate, ethyl butyrate, ethyl hexanoate, ethyl octanoate, ethyl decanoate, isobutyric acid, 2-methylbutyric acid, 3-methylbutyric acid, isovaleric acid, ethyl isobutyrate, ethyl 2-methylbutyrate, ethyl 3-methylbutyrate, ethyl isovalerate, carbonyl, lactone, diacetyl, 2,3-pentanedione, acetoine, γ-butyrolactone, ethyl lactate, diethyl succinate, Z-whiskylactone, E-whiskylactone, o and m-cresol, guaiacol, 4-methylguaiacol, eugenol, E-isoeugenol, 2,6-dimethoxyphenol, 4-allyl-2,6-dimethoxyphenol, vanillin, acetovanillone, propiovanillone, ethylvanillate, methylvanillate, furfural, 5-methylfurfural, 4-ethylphenol, 4-ethylguaiacol, 4-propylguaiacol, γ-lactones, γ-octalactone, γ-nonalactone, γ-decalactone, γ-undecalactone, γ-dodecalactone, 4-vinylphenol, 4-vinylguaiacol, or any combination of the foregoing.

111. The system of claim 106, wherein the plurality of samples comprises a grape product spiked with one or more compounds causing the one or more perceptual characteristics and/or a wine spiked with one or more compounds causing the one or more perceptual characteristics.

112. The system of claim 106, wherein the plurality of samples comprises a grape product spiked with a microbial organism.

113. The system of claim 106, wherein the optical signals from the samples comprise Raman spectra.

114. The system of claim 106, wherein the optical signals from the samples each comprise information from at least 10 wavelengths.

115. The system of claim 106, wherein the training data further comprises information from infrared spectra, fluorescence, and/or visible spectra of the grape produce or the wine.

116. The method of claim 106, wherein the processor and memory are configured to train the feature extractor by training a variational autoencoder or a transformer model using the optical signals generated from the samples.

117. The system of claim 106, wherein the machine learning model is a neural network.
